SEQUENCE LISTING (219 sequences)

SEQ ID NO: 1 - skipped

SEQ ID NO: 2 - DNA, 21 bp, Pichia sp.
atgtcggaaccaaacaccaag

SEQ ID NO: 3 - DNA, 25 bp, Pichia sp.
atgcccgtagattcttctcataaga

SEQ ID NO: 4 - DNA, 27 bp, Pichia sp.
atgtatatcccacctacaagtttcaac

SEQ ID NO: 5 - DNA, 20 bp, Pichia sp.
atgactactgctcccccaac

SEQ ID NO: 6 - DNA, 25 bp, Pichia sp.
atgtctcaaaggaagttacaacaag

SEQ ID NO: 7 - DNA, 21 bp, Pichia sp.
atggcacaagaaagaacagac

SEQ ID NO: 8 - DNA, 21 bp, Pichia sp.
atggataacgacgtatcgaca

SEQ ID NO: 9 - DNA, 21 bp, Pichia sp.
atgtctactcacggagttcag

SEQ ID NO: 10 - DNA, 21 bp, Pichia sp.
atgtttggagaagtgggttct

SEQ ID NO: 11 - DNA, 23 bp, Pichia sp.
atgagttcgactaatccatcagg

SEQ ID NO: 12 - DNA, 20 bp, Pichia sp.
atggaagaagcgttgcaacc

SEQ ID NO: 13 - DNA, 25 bp, Pichia sp.
atgtctgcaagtacttacagttttg

SEQ ID NO: 14 - DNA, 18 bp, Pichia sp.
atgacactctcccagggg

SEQ ID NO: 15 - DNA, 31 bp, Pichia sp.
atgaagttcattgatgaaattgatatagagc

SEQ ID NO: 16 - DNA, 23 bp, Pichia sp.
atgacacaagcagagttgtacac

SEQ ID NO: 17 - DNA, 25 bp, Pichia sp.
atgtcggattttcctaaagtcgaac

SEQ ID NO: 18 - DNA, 22 bp, Pichia sp.
atgatatccaacctctcggcag

SEQ ID NO: 19 - DNA, 27 bp, Pichia sp.
atggttcatatcttactcgatcaacac

SEQ ID NO: 20 - DNA, 20 bp, Pichia sp.
atggctgctgtatttttccc

SEQ ID NO: 21 - DNA, 27 bp, Pichia sp.
atgacactttttgataacctcaaacag

SEQ ID NO: 22 - DNA, 20 bp, Pichia sp.
atggcagaccaacggcttga

SEQ ID NO: 23 - DNA, 22 bp, Pichia sp.
atggctgctaggtctagtactc

SEQ ID NO: 24 - DNA, 25 bp, Pichia sp.
atgtctacaacaaaaccaatgcagg

SEQ ID NO: 25 - DNA, 20 bp, Pichia sp.
atggaagactcatccaaggc

SEQ ID NO: 26 - DNA, 22 bp, Pichia sp.
atggctaacctaaagatccctg

SEQ ID NO: 27 - DNA, 19 bp, Pichia sp.
atgcggcatggatttcaag

SEQ ID NO: 28 - DNA, 25 bp, Pichia sp.
atggtataccaatactcagtctctc

SEQ ID NO: 29 - DNA, 24 bp, Pichia sp.
atgacgctaaagatgaccaacttc

SEQ ID NO: 30 - DNA, 25 bp, Pichia sp.
atgatcatagtagacggcgaaaaat

SEQ ID NO: 31 - DNA, 21 bp, Pichia sp.
atgttccccgataataaacgg

SEQ ID NO: 32 - DNA, 25 bp, Pichia sp.
atgacatttcaagtagccttaccga

SEQ ID NO: 33 - DNA, 20 bp, Pichia sp.
atgaccactgcctatcccaa

SEQ ID NO: 34 - DNA, 25 bp, Pichia sp.
atgagtgacgtggtaaacaagagag

SEQ ID NO: 35 - DNA, 20 bp, Pichia sp.
atgtcgtccccaagaaagag

SEQ ID NO: 36 - DNA, 24 bp, Pichia sp.
atgaatcgacacgagatcagattg

SEQ ID NO: 37 - DNA, 25 bp, Pichia sp.
atgcctttctatcttcctgaagatg

SEQ ID NO: 38 - DNA, 23 bp, Pichia sp.
atgttgcccagaattttagcaag

SEQ ID NO: 39 - DNA, 20 bp, Pichia sp.
atgaactcagatcccacgtc

SEQ ID NO: 40 - DNA, 22 bp, Pichia sp.
atggataatccgtttggacagg

SEQ ID NO: 41 - DNA, 26 bp, Pichia sp.
atgctcgatgaagaaattctaacaag

SEQ ID NO: 42 - DNA, 28 bp, Pichia sp.
atgggtagaaggaaaatagagataaatc

SEQ ID NO: 43 - DNA, 20 bp, Pichia sp.
atgagcaatctacccccaac

SEQ ID NO: 44 - DNA, 23 bp, Pichia sp.
tcattctcccttgatggcttttc

SEQ ID NO: 45 - DNA, 25 bp, Pichia sp.
tcacctgatcgctatgcatgtcaac

SEQ ID NO: 46 - DNA, 25 bp, Pichia sp.
ttatatcttatctggcgtccaatcc

SEQ ID NO: 47 - DNA, 25 bp, Pichia sp.
tcaagagtccgagttcatgatattg

SEQ ID NO: 48 - DNA, 25 bp, Pichia sp.
tcaaagatacaacgtttccaacttg

SEQ ID NO: 49 - DNA, 24 bp, Pichia sp.
ttatgactctactgaattggctcc

SEQ ID NO: 50 - DNA, 30 bp, Pichia sp.
ctatacccaaatctcttttccagcattatc

SEQ ID NO: 51 - DNA, 20 bp, Pichia sp.
ctagagactcgaagttgcgc

SEQ ID NO: 52 - DNA, 27 bp, Pichia sp.
ctaatttgaatcaaactgatcagaagc

SEQ ID NO: 53 - DNA, 22 bp, Pichia sp.
ttatttctgaggttgggtagcc

SEQ ID NO: 54 - DNA, 26 bp, Pichia sp.
tcattcatctgttatctcaggctcag

SEQ ID NO: 55 - DNA, 25 bp, Pichia sp.
tcactgatgtttcactaaactcttt

SEQ ID NO: 56 - DNA, 28 bp, Pichia sp.
tcatttgatactgatttcgtagatcatc

SEQ ID NO: 57 - DNA, 22 bp, Pichia sp.
ttaactaacctgcaggtcttcc

SEQ ID NO: 58 - DNA, 22 bp, Pichia sp.
ttaccagaaagaacaccaaccg

SEQ ID NO: 59 - DNA, 25 bp, Pichia sp.
ctatttcttagccctcttactagca

SEQ ID NO: 60 - DNA, 21 bp, Pichia sp.
ctatgtatcaccacgtagggc

SEQ ID NO: 61 - DNA, 25 bp, Pichia sp.
ttatggtacactcatacccttcttg

SEQ ID NO: 62 - DNA, 20 bp, Pichia sp.
ttagttggcctcagcagttg

SEQ ID NO: 63 - DNA, 26 bp, Pichia sp.
tcagtatttagaagattggttgaccg

SEQ ID NO: 64 - DNA, 25 bp, Pichia sp.
tcagtcatctatgttctgaccaagc

SEQ ID NO: 65 - DNA, 22 bp, Pichia sp.
tcatcgcagatctatgtcaacc

SEQ ID NO: 66 - DNA, 25 bp, Pichia sp.
tcactgcttacggtgagtacgtaga

SEQ ID NO: 67 - DNA, 25 bp, Pichia sp.
tcagttggtcctcagaatctcctca

SEQ ID NO: 68 - DNA, 29 bp, Pichia sp.
ttactcatcatatatactactgtgcatga

SEQ ID NO: 69 - DNA, 22 bp, Pichia sp.
ctagtaaccatcgtgttcctgc

SEQ ID NO: 70 - DNA, 25 bp, Pichia sp.
ttattcttcaaaccctttggtttcc

SEQ ID NO: 71 - DNA, 32 bp, Pichia sp.
ctaatagagagtaccaaaatgagtttgatatc

SEQ ID NO: 72 - DNA, 25 bp, Pichia sp.
ttacaagggcagagatttacaggtg

SEQ ID NO: 73 - DNA, 28 bp, Pichia sp.
ttacaaatctctgaaagaagaacctttg

SEQ ID NO: 74 - DNA, 30 bp, Pichia sp.
ctaaaggtattctacatgatcttttgcata

SEQ ID NO: 75 - DNA, 30 bp, Pichia sp.
tcatttcttttcttgattttccagagaggt

SEQ ID NO: 76 - DNA, 30 bp, Pichia sp.
ctatttaaacatggaaaaatcgacaacatc

SEQ ID NO: 77 - DNA, 21 bp, Pichia sp.
ctagttcttgaagcctttggc

SEQ ID NO: 78 - DNA, 28 bp, Pichia sp.
tcattcagggagtaactgatcaataatc

SEQ ID NO: 79 - DNA, 30 bp, Pichia sp.
tcacttcttgtaattgttaaaaatatggtc

SEQ ID NO: 80 - DNA, 26 bp, Pichia sp.
ttatttatcggtgtcacttccatctg

SEQ ID NO: 81 - DNA, 27 bp, Pichia sp.
ttagaattcatacactgacttttcagg

SEQ ID NO: 82 - DNA, 21 bp, Pichia sp.
ttaatgcgtcgaatgtcctcg

SEQ ID NO: 83 - DNA, 24 bp, Pichia sp.
ctagataacaggcacagtttttcg

SEQ ID NO: 84 - DNA, 22 bp, Pichia sp.
tcagctcttcttagtcacactg

SEQ ID NO: 85 - DNA, 22 bp, Pichia sp.
ctagacaccaccatctagtcgg

SEQ ID NO: 86 - DNA, 25 bp, Pichia sp.
cattctaaaggccgattttttcagg

SEQ ID NO: 87 - DNA, 33 bp, Pichia sp.
ttattgtaagtgactaaagttgattttctattg

SEQ ID NO: 88 - DNA, 16 bp, Pichia sp.
caagcttggcccgcac

SEQ ID NO: 89 - DNA, 16 bp, Pichia sp.
aaggatactccgcgcg

SEQ ID NO: 90 - DNA, 21 bp, Pichia sp.
agactgctgctcagttagtag

SEQ ID NO: 91 - DNA, 21 bp, Pichia sp.
gtaaccaacccgaacgtatcg

SEQ ID NO: 92 - DNA, 22 bp, Pichia sp.
ggattctccaaccccttcaatc

SEQ ID NO: 93 - DNA, 24 bp, Pichia sp.
gaggtcaatggtcagttttctatg

SEQ ID NO: 94 - DNA, 23 bp, Pichia sp.
tatggcaaagcgtcagtaaaaag

SEQ ID NO: 95 - DNA, 17 bp, Pichia sp.
agggtctgagccaatgg

SEQ ID NO: 96 - DNA, 22 bp, Pichia sp.
gtcctgtgtatccaaccattga

SEQ ID NO: 97 - DNA, 34 bp, Pichia sp.
gctttttattgaaaattgtgttgttaaaatattc

SEQ ID NO: 98 - DNA, 24 bp, Pichia sp.
cttgagatccttagttagccaatg

SEQ ID NO: 99 - DNA, 20 bp, Pichia sp.
tttccacaagaacgaaaccc

SEQ ID NO: 100 - DNA, 21 bp, Pichia sp.
tactgtgattgactcgggaga

SEQ ID NO: 101 - DNA, 31 bp, Pichia sp.
aaaaaagttctgtgtttatattcagtttatg

SEQ ID NO: 102 - DNA, 23 bp, Pichia sp.
tcttggtgatattctgagaccaa

SEQ ID NO: 103 - DNA, 33 bp, Pichia sp.
atttcaataactgacatgtctttaataatcaag

SEQ ID NO: 104 - DNA, 23 bp, Pichia sp.
ccaacttagacctctcaaacaag

SEQ ID NO: 105 - DNA, 20 bp, Pichia sp.
acttctgacacacagtcctc

SEQ ID NO: 106 - DNA, 27 bp, Pichia sp.
gaaaatagtgaccaatctgtcatactc

SEQ ID NO: 107 - DNA, 23 bp, Pichia sp.
tcctgaaaaatggaaaggtcttc

SEQ ID NO: 108 - DNA, 19 bp, Pichia sp.
gctcagtggctttccatct

SEQ ID NO: 109 - DNA, 24 bp, Pichia sp.
tgacattgcaattcaattggactg

SEQ ID NO: 110 - DNA, 27 bp, Pichia sp.
ggaattatcatggagagaattgacaag

SEQ ID NO: 111 - DNA, 18 bp, Pichia sp.
ggatttcttgagggggct

SEQ ID NO: 112 - DNA, 20 bp, Pichia sp.
taatacggcaccgattgtcc

SEQ ID NO: 113 - DNA, 35 bp, Pichia sp.
atgataactttaatttttcatcctgtttatatacg

SEQ ID NO: 114 - DNA, 23 bp, Pichia sp.
ctgtattttcagtcatttggtcg

SEQ ID NO: 115 - DNA, 20 bp, Pichia sp.
catacgtggttgccatgaac

SEQ ID NO: 116 - DNA, 26 bp, Pichia sp.
gaagtattcaaaatgctagctcagtg

SEQ ID NO: 117 - DNA, 21 bp, Pichia sp.
ttcttggacttttatgagggc

SEQ ID NO: 118 - DNA, 21 bp, Pichia sp.
cggacaccatctatctcttca

SEQ ID NO: 119 - DNA, 25 bp, Pichia sp.
gagcagagtgatgaaagttttcaac

SEQ ID NO: 120 - DNA, 25 bp, Pichia sp.
gatataaaattggcaccactttctg

SEQ ID NO: 121 - DNA, 19 bp, Pichia sp.
taccacctcgtggtttgac

SEQ ID NO: 122 - DNA, 27 bp, Pichia sp.
tcatgatataattgatcaagatttgcg

SEQ ID NO: 123 - DNA, 20 bp, Pichia sp.
aatttgcctaagccaaggac

SEQ ID NO: 124 - DNA, 26 bp, Pichia sp.
tgaagaagagattgttgagattttgg

SEQ ID NO: 125 - DNA, 23 bp, Pichia sp.
cggatacagctattgttacctct

SEQ ID NO: 126 - DNA, 23 bp, Pichia sp.
tattgaaggaggtgacaaagctg

SEQ ID NO: 127 - DNA, 27 bp, Pichia sp.
aattgtggatcttatctatagcaaggc

SEQ ID NO: 128 - DNA, 24 bp, Pichia sp.
atgttggtattgtgaaatagacgc

SEQ ID NO: 129 - DNA, 22 bp, Pichia sp.
ggtcatgcagatcctcatgttc

SEQ ID NO: 130 - DNA, 27 bp, Pichia sp.
ttgaaaaagaagtttttttctcacaag

SEQ ID NO: 131 - DNA, 28 bp, Pichia sp.
attatgcattaatcaggaaaattcacat

SEQ ID NO: 132 - DNA, 17 bp, Pichia sp.
tttccactggagcgtgc

SEQ ID NO: 133 - DNA, 26 bp, Pichia sp.
tgatgatcttgattttcatgttttcc

SEQ ID NO: 134 - DNA, 18 bp, Pichia sp.
cacccgaagacgaacaga

SEQ ID NO: 135 - DNA, 23 bp, Pichia sp.
actaataatgaaacgcccaaact

SEQ ID NO: 136 - DNA, 22 bp, Pichia sp.
ctatgttgacggagagtgttgg

SEQ ID NO: 137 - DNA, 17 bp, Pichia sp.
ctgcggaagagttggcg

SEQ ID NO: 138 - DNA, 31 bp, Pichia sp.
tagtttaggctcgaaattaattagtaattag

SEQ ID NO: 139 - DNA, 19 bp, Pichia sp.
gtagcagactttggggacc

SEQ ID NO: 140 - DNA, 23 bp, Pichia sp.
ataatagttggatgggatctggc

SEQ ID NO: 141 - DNA, 28 bp, Pichia sp.
catattcaaataccaagcaaataaacgc

SEQ ID NO: 142 - DNA, 24 bp, Pichia sp.
gattgctccagaaaaatgttggat

SEQ ID NO: 143 - DNA, 21 bp, Pichia sp.
cttggggaacacagttgaaag

SEQ ID NO: 144 - DNA, 25 bp, Pichia sp.
aatgaggacgatggtaacaaaactc

SEQ ID NO: 145 - DNA, 28 bp, Pichia sp.
atacagtttgatgatgaagagttcataa

SEQ ID NO: 146 - DNA, 16 bp, Pichia sp.
ggccgccactcagatc

SEQ ID NO: 147 - DNA, 25 bp, Pichia sp.
agttttgtttggaggataacaagtc

SEQ ID NO: 148 - DNA, 22 bp, Pichia sp.
cggctcgtttgattctacaatc

SEQ ID NO: 149 - DNA, 18 bp, Pichia sp.
ccaatccagcagacaccc

SEQ ID NO: 150 - DNA, 21 bp, Pichia sp.
aggataggaggggtgaattcc

SEQ ID NO: 151 - DNA, 19 bp, Pichia sp.
atgtgaaggcaaggggttc

SEQ ID NO: 152 - DNA, 20 bp, Pichia sp.
caaagaagaagccaccatcg

SEQ ID NO: 153 - DNA, 22 bp, Pichia sp.
ctctttgatgattggtgttggg

SEQ ID NO: 154 - DNA, 24 bp, Pichia sp.
tttctgcgatctgattcgactaag

SEQ ID NO: 155 - DNA, 21 bp, Pichia sp.
tgggtgattctggtttgtttg

SEQ ID NO: 156 - DNA, 23 bp, Pichia sp.
tgcgaagtatctattggtgactg

SEQ ID NO: 157 - DNA, 23 bp, Pichia sp.
gtgtgtggtggttaagaactgtc

SEQ ID NO: 158 - DNA, 28 bp, Pichia sp.
tttctctatgaataggctaggaaaattc

SEQ ID NO: 159 - DNA, 28 bp, Pichia sp.
ctatgttttagtgtaagaaggtctttgc

SEQ ID NO: 160 - DNA, 23 bp, Pichia sp.
ctgagacggcaaaaacttttttc

SEQ ID NO: 161 - DNA, 24 bp, Pichia sp.
gagggtcttttcaaaggtccaatg

SEQ ID NO: 162 - DNA, 29 bp, Pichia sp.
ctaaacgaaaacaaatgaacaaacaaatg

SEQ ID NO: 163 - DNA, 24 bp, Pichia sp.
tgttctgttgctttcctaaaactc

SEQ ID NO: 164 - DNA, 28 bp, Pichia sp.
cactgatgtttcactaaactctttaaac

SEQ ID NO: 165 - DNA, 16 bp, Pichia sp.
atggcacgcggactag

SEQ ID NO: 166 - DNA, 21 bp, Pichia sp.
tgtagtgaacgtagcatgcac

SEQ ID NO: 167 - DNA, 19 bp, Pichia sp.
agggatacagcagaggacg

SEQ ID NO: 168 - DNA, 23 bp, Pichia sp.
tgtattgcgggattataatgtcc

SEQ ID NO: 169 - DNA, 25 bp, Pichia sp.
gggtttaaatggaagcagaaaagtg

SEQ ID NO: 170 - DNA, 18 bp, Pichia sp.
gattggggaggcgaagag

SEQ ID NO: 171 - DNA, 28 bp, Pichia sp.
cttgtaaggggaaaaaaattattggatg

SEQ ID NO: 172 - DNA, 26 bp, Pichia sp.
aatgagccgaatattcccttcaaatc

SEQ ID NO: 173 - DNA, 19 bp, Pichia sp.
gtttacgctccgttcaggg

SEQ ID NO: 174 - DNA, 30 bp, Pichia sp.
tttattatggttccctaacaaattctatgg

SEQ ID NO: 175 - DNA, 25 bp, Pichia sp.
ctagttaatcgcaaacaaacccaaa

SEQ ID NO: 176 - DNA, 20 bp, Pichia sp.
cctctgtagtgggtaggtgg

SEQ ID NO: 177 - DNA, 27 bp, Pichia sp.
gtatggtcgtatagagatatacgagag

SEQ ID NO: 178 - DNA, 27 bp, Pichia sp.
tgttccaatttttctaaatataggcca

SEQ ID NO: 179 - DNA, 21 bp, Pichia sp.
ccagctgtatattaaaggcgg

SEQ ID NO: 180 - DNA, 21 bp, Pichia sp.
aattcagtggtttgtgagcaa

SEQ ID NO: 181 - DNA, 21 bp, Pichia sp.
aacactgtatccctgtatccc

SEQ ID NO: 182 - DNA, 21 bp, Pichia sp.
cctgactaacggtgtatgtga

SEQ ID NO: 183 - DNA, 24 bp, Pichia sp.
ggttgatgtagtttgtagtggatg

SEQ ID NO: 184 - DNA, 33 bp, Pichia sp.
gataaaaaaaattttatggtgttcagtttacag

SEQ ID NO: 185 - DNA, 20 bp, Pichia sp.
tgctgtttgtgtgattgttg

SEQ ID NO: 186 - DNA, 24 bp, Pichia sp.
ggtgactttttgtgaaatgatctg

SEQ ID NO: 187 - DNA, 25 bp, Pichia sp.
tatggatagatttagtgacaggagc

SEQ ID NO: 188 - DNA, 21 bp, Pichia sp.
tctctggaattcatctccgtg

SEQ ID NO: 189 - DNA, 20 bp, Pichia sp.
cctggggtaatcattgggaa

SEQ ID NO: 190 - DNA, 29 bp, Pichia sp.
attgtgtaagtaagatgaaaaacaatttc

SEQ ID NO: 191 - DNA, 24 bp, Pichia sp.
ttgaagcaagctgtaatagaagtg

SEQ ID NO: 192 - DNA, 22 bp, Pichia sp.
gattggggagtgggaatactta

SEQ ID NO: 193 - DNA, 21 bp, Pichia sp.
tgtcgattctaaaggtggtgt

SEQ ID NO: 194 - DNA, 21 bp, Pichia sp.
tgtgcgtgggataaagtcatc

SEQ ID NO: 195 - DNA, 28 bp, Pichia sp.
cgtttcgaataattagttgttttttgat

SEQ ID NO: 196 - DNA, 21 bp, Pichia sp.
taacaggcacctgaagatagg

SEQ ID NO: 197 - DNA, 26 bp, Pichia sp.
ggttttgtatgatgaagaggtaaaac

SEQ ID NO: 198 - DNA, 25 bp, Pichia sp.
ttttatttgacgattcagatggtgc

SEQ ID NO: 199 - DNA, 22 bp, Pichia sp.
ctcgtaggtgaaagatcggatg

SEQ ID NO: 200 - DNA, 25 bp, Pichia sp.
tatcagttgaataagtgtcagtgac

SEQ ID NO: 201 - DNA, 29 bp, Pichia sp.
tgtcgaataaagacgttattaatcgaatg

SEQ ID NO: 202 - DNA, 21 bp, Pichia sp.
cttgtagtcttattggggggt

SEQ ID NO: 203 - DNA, 28 bp, Pichia sp.
tttaattgtaagtcttgactagagcaag

SEQ ID NO: 204 - DNA, 26 bp, Pichia sp.
tgctattagtttctgtgattctgttc

SEQ ID NO: 205 - DNA, 31 bp, Pichia sp.
tatcttaatctagctattgctttgaaaatga

SEQ ID NO: 206 - DNA, 22 bp, Pichia sp.
tttatggttggtgaagtcttgg

SEQ ID NO: 207 - DNA, 29 bp, Pichia sp.
gctaatgtaaaaagtaagattgctttcaa

SEQ ID NO: 208 - DNA, 27 bp, Pichia sp.
tgttgtgtactagctaattgattgatg

SEQ ID NO: 209 - DNA, 30 bp, Pichia sp.
cgttaaacaaaaaaaattgttttcaatgag

SEQ ID NO: 210 - DNA, 26 bp, Pichia sp.
tgtttggtttttactagttttctggt

SEQ ID NO: 211 - DNA, 30 bp, Pichia sp.
gaataggatttgatggaaagaataaaactg

SEQ ID NO: 212 - DNA, 23 bp, Pichia sp.
tttggtgatgagtttttatgggg

SEQ ID NO: 213 - DNA, 18 bp, Pichia sp.
tgggtttgagagttgggg

SEQ ID NO: 214 - DNA, 23 bp, Pichia sp.
cctattgggggtatcatacagag

SEQ ID NO: 215 - DNA, 20 bp, Pichia sp.
cgtttgacggagaagctgtt

SEQ ID NO: 216 - DNA, 26 bp, Pichia sp.
tgtgatttaccagagataagtatgga

SEQ ID NO: 217 - DNA, 22 bp, Pichia sp.
gatatggtgggagagctgatag

SEQ ID NO: 218 - DNA, 23 bp, Pichia sp.
gatgcgtttggagtttagatgaa

SEQ ID NO: 219 - DNA, 24 bp, Pichia sp.
gggaacaatgaaggaactgaaaag

(19) United States
(12) Patent Application Publication
(10) Pub. No.: US 2015/0284711 A1
(43) Pub. Date: Oct. 8, 2015

(54) USE OF A REWIRED GENETIC CONTROL NETWORK TO INCREASE PRODUCT EXPRESSION

(71) Applicant: IMPERIAL INNOVATIONS LIMITED, South Kensington, London (GB)

(72) Inventors: Travis Scott Bayer, London (GB); Rui Tiago De Lima Rodrigues, London (GB); Oliver Peter Foster Windram, West Drayton (GB)

(73) Assignee: IMPERIAL INNOVATIONS LIMITED, London (GB)

(21) Appl. No.: 14/443,270

(22) PCT Filed: Nov. 15, 2013
(86) PCT No.: PCT/GB2013/053031

§ 371 Date: May 15, 2015

(30) Foreign Application Priority Data
Nov. 16, 2012 (GB) 1220686.8

(51) Int. Cl. C12N 15/10

(52) U.S. Cl. CPC C12N 15/1058

(57) ABSTRACT

A method of identifying a cell with increased production of a desired product, the method comprising: (a) providing a population of cells which produce the desired product; (b) introducing a plurality of distinct nucleic acid molecule species into the population of cells, wherein the nucleic acid molecules comprise a first region which is operably-linked to a second region, wherein the first region comprises a promoter region from a first gene, and the second region comprises a nucleic acid sequence from a second gene that is capable of expressing a regulatory product under control of the promoter region; (c) culturing the population of cells under conditions in which the cells express the regulatory product under control of the promoter region; and (d) testing the population of cells for production of the desired product, thereby to identify a cell or cells with increased production of the desired product. The method may further comprise isolating a cell or cells with increased production of the desired product. The method may further comprise repeating steps (b), (c) and (d) on the isolated cells or cells, or descendants thereof with increased production of the desired product, thereby to identify a cell or cells with further increased production of the desired product. Typically the desired product is not encoded by the introduced nucleic acid molecule. The plurality of distinct nucleic acid molecule species typically comprise a plurality of combinations of multiple first and multiple second regions. The expressed regulatory product typically is a polypeptide but may be an RNA molecule. The method may further comprise isolating the nucleic acid molecule(s) introduced into the cell or cells with increased production of the desired product.

The present invention relates to methods useful in enhancing production of cellular products.

The demand for industrial proteins and metabolites is growing across market sectors as diverse as health care, food and beverage processing, crop and animal protection, and nutritional and personal care products. One of the significant challenges for the manufacture of bioproducts is the low yields that microbial cells produce—a major research and development activity of bioproduct companies is improving strain yield and productivities.

### Engineering Microbial Cell Factories

The use of microbes for production of industrial and consumer products is increasingly important in many industries. In protein production yeast represent useful eukaryote single cell expression factorise with the potential to express foreign proteins with complex post-translational modifications such as glycosylation, disulfide bond formation, phosphorylation and proteolytic processing. Host species such as Saccharomyces cerevisiae and Pichia pastoris are currently used as industrial scale protein expression platforms for enzymes, biopharmaceuticals, and food additives.

Several groups have engineered microbes to produce fine chemicals for therapeutic purposes. A group at UC Berkeley has engineered Saccharomyces cerevisiae to produce artemisinic acid, a precursor to artemisinin, a potent anti-malarial naturally found in the plant Artemisia annua. Currently, the chemical synthesis of artemisinin is cost prohibitive to the population of the developing world, where it is needed most. The researchers were able to use an engineered mevalonate pathway, an amorphadiene synthase, and a cytochrome P450 monooxygenase from A. annua to produce artemisinic acid. The engineered yeast produced higher artemisinin yields than A. annua, although the authors note that industrial scale-up and strain optimization will be required to make this route to production cost-effective.

### Current State of Metabolic Optimisation Strategies

Traditionally the optimisation of yield of bio-products relied on manipulating process conditions in the fermenter, such as feed rate of carbon source, temperature, pH and media composition. Modern metabolic engineering relies on genetic changes to host strains, for example gene knockouts or overexpression to enhance metabolic flux toward the pathway and/or product of interest. These genetic changes can be predicted by mathematical models of metabolism (ie flux balance models) as well as biological intuition (for example, deleting enzymes that catalyze undesirable side reactions). In industry (as well as academia), selection and optimisation of “production strains” is largely a trial-and-error process, requiring significant time and resource investments to increase product yields or develop strains for new products.

### Rewiring Genetic Control Networks for Enhanced Product Yields

Synthetic rewiring of gene regulation has been used in E. coli to study the robustness and evolvability of the natural regulatory network (Isalan et al (2008) “Evolvability and hierarchy in rewired bacterial gene networks” Nature 452, 840-846). In this study the authors constructed a library of transcription factor and sigma factor open reading frames (ORFs) under the control of regulatory promoters from different genes. Isalan et al constructed and screened approximately 600 such rewiring and found that 98 percent resulted in viable cells. This included rewiring the expression of global regulators of gene expression such as sigma 70, which controls the expression of approximately 1000 genes. While Isalan showed that the majority of rewirings are tolerated (in terms of effect on growth rate) they did not test or speculate on the ability of rewired strains to overproduce protein or metabolite products.

The listing or discussion of an apparently prior-published document in this specification should not necessarily be taken as an acknowledgement that the document is part of the state of the art or is common general knowledge. Any document referred to herein is hereby incorporated by reference.

Here, we adopted a novel approach to metabolic optimization that relies on the reprogramming of the cellular response to potential “overproduction stress” rather than flux balance models or gene knockout/overexpression efforts. Heterologous protein expression and/or metabolite production presents a highly unnatural cellular state that invokes a number of response which can have adverse effects on quality and quantity of the desired product. For instance, accumulation of mis-folded protein in cellular compartments can invoke an unfolded protein stress response leading to a reduction in production, while hyperglycosylation can also produce unfavourable by-products. Induction of many of these deleterious processes often possess a transcriptional element. Thus, we considered that reprogramming the host transcriptome could circumvent or repurpose these events to favour productivity.

We have developed a platform technology we call genetic rewiring that we consider significantly improves strain performance in a rapid manner, and have successfully tested this method on a variety of industrially relevant proteins and metabolites. We consider that we have demonstrated the application of the platform technology to exemplar bioproducts for commercial exploitation, as well as developing predictive design and analysis tools underlying the performance of the platform.

A first aspect of the invention provides a method of identifying a cell with increased production of a desired product, the method comprising:
(a) providing a population of cells which produce the desired product;
(b) introducing a plurality of distinct nucleic acid molecule species into the population of cells, wherein the nucleic acid molecules comprise a first region which is operably-linked to a second region, wherein
   the first region comprises a promoter region from a first gene, and
   the second region comprises a nucleic acid sequence from a second gene that is capable of expressing a regulatory product under control of the promoter region;
(c) culturing the population of cells under conditions in which the cells express the regulatory product under control of the promoter region; and
(d) testing the population of cells for production of the desired product, thereby to identify a cell or cells with increased production of the desired product.

Typically the desired product is not encoded by the introduced nucleic acid molecule.

Host organisms in protein and metabolite production bioprocesses are subject to a number of dynamic stresses and conditions in the bioreactor and processing environment. Conditions such as low oxygen, changing pH, shear stress, and nutrient limitation induce physiological states in the host cell that are suboptimal for the overproduction of active products. The rewiring platform entails construction of a library of nucleic acid molecule species as set out above, for example comprising transcription factor open reading frames (ORFs) (examples of “nucleic acid sequence from a second gene that is capable of expressing a regulatory product under control of the promoter region”) under the control of regulatory promoters from different genes. These synthetic promoter-regulatory product-expressing (for example ORF) constructs act as rewiring elements when introduced into the cell: for example, environmental conditions that induce the expression of a given gene (in the wildtype organism) now also induce the expression of an unrelated regulatory gene. This has the effect of randomly placing links between different nodes of the network. As examples, the library may be constructed using transcription factors implicated in control of ribosome biogenesis, stress response, stationary phase, heat shock, and the unfolded protein response. In addition, the library may include the promoters of genes induced by a variety of dynamic bioreactor conditions, including dissolved oxygen, pH, carbon source, and temperature. A promoter-regulatory product-expressing/ORF library can be created for virtually any transformable organism. These libraries can then be screened for enhanced production using, for example, fluorescent readouts or high-throughput screening platforms.

The rewiring technology has been used to improve production and secretion of several proteins that are considered industrially relevant and difficult to produce: for example a human opioid receptor, insulin, an antibody fragment, a spider silk, and an immunotoxin therapeutic. Productivities are improved by over an order of magnitude compared to the parent production strain. In metabolite production, preliminary trials with yeast engineered to produce lycopene, for example, have shown productivities several fold greater than previously published results.

The method may further comprise isolating a cell or cells with increased production of the desired product. The method may further comprise repeating steps (b), (c) and (d) on the isolated cells or cells, or descendants thereof with increased production of the desired product, thereby to identify a cell or cells with further increased production of the desired product. This method may further comprise isolating a cell or cells with further increased production of the desired product.

It is considered that the iterative method, in which further a further plurality of distinct nucleic acid molecule species is introduced into cells which have been selected based on increased production of the desired product is particularly beneficial in obtaining useful increased production of the desired product.

It is considered that an increase of at least about 1.5-fold, 2-fold, 3-fold, 4-fold, 5-fold, 6-fold, 7-fold, 8-fold, 9-fold and 10-fold in expression level may be achieved using the methods of the invention, particularly when the “iterative” approach is used.

It will be appreciated that the further plurality of distinct nucleic acid molecule species may be selected without knowledge of (or without taking into account) the nature of the nucleic acid molecule species that has/have resulted in increased production of the desired product in the previous “round” of selection; or alternatively, the further plurality of distinct nucleic acid molecule species may be selected taking into account the promoter region and/or the regulatory product identified (as discussed further below) in designing a further “library”. Thus, the further plurality of distinct nucleic acid molecule species may be a further portion from the same “library” as the preceding plurality of distinct nucleic acid molecule species; or may be from a different “library”, for example to constructed using promoter region and/or regulatory product portions that are selected as related (for example in terms of the stimuli responded to or pathways controlled) to the identified promoter region(s) and/or identified regulatory product(s).

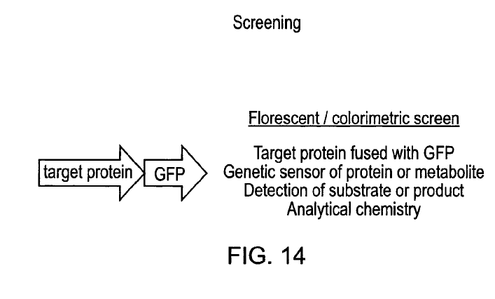

Methods for isolating a cell or cells with increased production of the desired product, or further increased production of the desired product, as appropriate, will be well known to those skilled in the art. For example, cells may be assessed on production of a fluorescent product (for example a product tagged with a fluorescent moiety, for example a GFP or similar), for example using Fluorescence Activated Cell Sorting (FACS) or using other, typically medium-high-throughput screening or sorting techniques. As will be well known to those skilled in the art, production may be assessed using, for example, enzymatic activity of the expressed product (for example using a chromogenic substrate) or using a specific binding partner, for example an antibody or antibody domain, or similar, as well known to those skilled in the art. Examples are also given in FIG. 14.

As an example, clones may be grown and assessed in microtitre plates, for example 96-well microtitre plates, for example as described in the Examples.

The level at which “increased production” of the desired product is deemed to have arisen will typically be selected by the skilled person. For example samples which show repeatable expression measurement greater than two standard deviations from the mean value may be selected/isolated, for example as described in the Examples. It will be appreciated that different parameters may be selected based on the requirements for a particular project. The comparison may be made with cells which have not been transformed with a distinct nucleic acid molecule species as set out above; or for convenience may be made with other cells which have been transformed with such a distinct nucleic acid molecule species as set out above, for example by taking a mean of the values obtained across a series of samples, for example across a microtitre plate.

Typically, the plurality of distinct nucleic acid molecule species comprises a plurality of combinations of multiple first and multiple second regions. Thus, for example, the “library” comprising the plurality of distinct nucleic acid molecule species may comprise two or more first regions each combined with each of two or more second regions. Thus, for example, at least 5, 10, 15, 20, 30, 40, 50, 60, 70, 80, 90 or 100 first (promoter) regions may be combined with at least 5, 10, 15, 20, 30, 40, 50, 60, 70, 80, 90 or 100 second (regulatory product) regions. In one example, as described further in the Examples, 43 open reading frames of transcriptional regulators were randomly combined with 67 promoters of genes involved in signalling, transport and carbon metabolism. It will be appreciated that the number of promoter regions and number of regulatory product regions combined, and hence the size of the library, will be selected based upon the requirements of the project, for example the ease with which high throughput screening for expression of the desired product can be performed. The larger the library, the more screening required, but the wider the range of combinations that can be assessed and potentially the greater the chance of identifying a useful combination. Typically the number of promoter regions and the number of regulatory product regions combined is each less than 200. However, the number could be higher, for example if an essentially “minimally selected” or “all-inclusive” approach is taken to forming the library, as discussed further below.

The expressed regulatory product is typically a polypeptide. Alternatively, the expressed regulatory product may be an RNA molecule, as will be well known to those skilled in the art.

The expressed regulatory product typically is a product that has been identified (or is identifiable, for example through sequence similarity or through other interactions, as discussed further below) as involved in regulation of gene expression, typically regulation of expression of a gene or genes other than the gene for the product itself. The expressed regulatory product may however be any gene product, for example when seeking to form the widest possible library to be screened (for example when using a “minimally selected” or “all-inclusive” approach. The gene product may thus be a potential or identified regulatory product. Typically the expressed regulatory product is a gene product from the same phylum, genera, species or strain as the population of cells to which the method is applied. Examples of regulatory product that can be included are discussed further below, for example.

The term “promoter region” will be well known to those skilled in the art. Typically, a promoter region is a nucleic acid region that has been identified (or is identifiable, for example through sequence similarity) as sufficient for expression of a neighbouring coding region, for example regulated expression of a neighbouring coding region, as will be well known to those skilled in the art. Typically the promoter is a nucleic acid sequence involved in the binding of RNA polymerase to initiate transcription of a gene. Typically the promoter region is a promoter region from the same phylum, genera, species or strain as the population of cells to which the method is applied. Examples of promoter regions that can be included are discussed further below, for example. The promoter region may however be any gene region, for example when seeking to form the widest possible library to be screened (for example when using a “minimally selected” or “all-inclusive” approach. The promoter region may thus be a potential or identified promoter region.

The method may further comprise isolating the nucleic acid molecule(s) introduced into the cell or cells with increased production of the desired product. Suitable methods for isolating the nucleic acid molecule(s) will be well known to those skilled in the art, for example following isolation of the cell or cells with increased production of the desired product, as noted above. Nucleic acid amplification techniques, for example Polymerase Chain Reaction (PCR) may be used, as well known to those skilled in the art, and as described, for example, in the Examples.

A further aspect of the invention provides a method of producing a cell with increased production of a desired product, the method comprising:
providing a cell which produces a desired product; and
introducing into the cell a nucleic acid molecule that has been isolated as set out above.

The method of the first aspect of the invention may further comprise identifying the first and second regions of the nucleic acid molecule(s) introduced into the cell or cells with increased production of the desired product.

A further aspect of the invention provides a method of producing a cell with increased production of a desired product, the method comprising:
providing a cell which produces a desired product; and
introducing into the cell a nucleic acid molecule comprising a promoter region operably-linked to a nucleic acid sequence that is capable of expressing a regulatory product under control of the promoter region, wherein the promoter region, and the nucleic acid sequence that is capable of expressing the regulatory product, have been identified as set out above.

Thus, the nucleic acid introduced into the cell may comprise the identified promoter region and the identified nucleic acid sequence that is capable of expressing the regulatory product.

In some instances, a regulatory product may be isolated without an associated promoter region (or a promoter region may be isolated without an associated regulatory product region). In such cases, the isolated regulatory product region (or promoter region) may be introduced into the cell.

A further aspect of the invention provides a method of producing a desired product, the method comprising:
culturing a cell with increased production of the desired product that has been produced, identified or isolated according to the method of any of the preceding aspects of the invention, or the descendants thereof with increased production of the desired product, under conditions in which the cell expresses the desired product.

The method may further comprise recovering the desired product from the cells or the cell culture. The method may further comprise isolating, concentrating, purifying and/or formulating the desired product. Techniques appropriate for the particular product will be known to those skilled in the art and may include techniques such as chromatographic separation or affinity purification.

In any of the preceding aspects of the invention the cell may be a prokaryotic cell. Examples of such cells will be well known to those skilled in the art and include Gram negative bacteria; Gram positive bacteria; and mycobacteria or acid fast bacteria, for example Mycobacterium smegmatis. Examples of Gram-negative bacteria include E. coli, S. enterica, for example S. entericia serovar Typhimurium, Salmonella spp and Campylobacter spp. Examples of Gram-positive bacteria include S. aureus.

Illustrative examples of cells and products which have been produced in those cells (which production, for example, may be increased using methods of the invention) include the following:

TABLE 1
examples of prokaryotic cells and typical products.

| Prokaryotes | Products |
|---|---|
| Acetobacterium | ethanol |
| Acinetobacter | L-carnitine |
| Actinobacillus | Succinic acid, butadiene |
| Aerobacter | amylo-1,6-glucosidase, pullulanase, citric acid, butylene, |
| | 1,3-propanediol |
| Alcaligenes | lipase, heteroaromatic carboxylic acids, polyhydroxyalkanoate, |
| | 6-hydroxypicolinic acid, L-aminoacylase, phb |
| Algibacter | zeaxhantin, carotenoids |
| Alteromonas | thiomarinol, eicosapentaenoic acid, bisucaberin |
| Anabaena | ethanol |
| Anaerobiospirillum | Succinic acid, butadiene |
| Arthrobacter | trehalose, L-phenylalanine |
| Bacillus | purines/pyrimidines, NAD, AMP, butanediol, riboflavin, enzymes, |
| | butadiene, citric acid, 1,3-propanediol, L-phenylalanine |
| Brevibacterium | trehalose |
| Butyribacterium | ethanol |
| Citrobacter | Phytase, 2-keto-L-gluconic acid, 1,3-propanediol, |
| | L-phenylalanine |
| Clostridium | propionic acid, butyric acid, butanol, ethanol, butadiene, |
| | 1,3-propanediol |
| Comamonas | acrilic acid, methacylic acid, 2-hydroxyisobutyric, |
| | N-acylproline-acylase, |
| | 3-hydroxycarboxylic acid, 3-hydroxyvaleric acid |
| Corynobacterium | Diaminopimelic acid, lysine, glutamate, methionin, |
| | phenylalanin, trehalose, |
| | butadiene |
| Cyclotella | |
| Delftia | |
| Enterobacter | butanediol, 1,3-propanediol, L-phenylalanine |
| Escherichia | threonin, aspartic acid, phenylalanin, succinic acid, |
| | propanediol, adipic acid, enzymes, alkane, alkene, alcohols, |
| | charotenoids, butadiene, |
| | rifamycin, 1,3-propanediol |
| Eubacterium lymosum | ethanol |
| Euglena gracilis | |
| Flavobacterium | L-phenylalanine |
| Gluconobacter | ascorbic acid, butadiene |
| Haemotacoccus | |
| Klebsiella | butanediol, butadiene, 1,3-propanediol, L-phenylalanine |
| Lactobacillus | tartaric acid, hydroxypropionic acid, lactic acid, butadiene, |
| | 1,3-propanediol |
| Methanobacterium | |
| Methylobacter | 1,3-propanediol |
| Methylomonas | |
| Mycobacterium | trehalose, L-phenylalanine |
| Nitzschia | |
| Pandorea | |
| Pantoa | |
| Paracoccus | L-phenylalanine |
| peptostreptococcus | ethanol |
| Propionibacterium | propionic acid, B12 |
| Pseudomonas | B12, coronatine, rifamycin, phenazine, pyrrolnitrin, citric acid, |
| | 1,3-propanediol, L-phenylalanine |
| Pyrococcus | |
| Rhizobium | B6, butadiene |
| Rhodococcus | amides |
| Rhodospirillum | ethanol |
| Rhodpseudomonas | Eicopentaenoic acid, docosahexaenoic acid, ethanol |
| Salmonella | 1,3-propanediol, L-phenylalanine |
| Shewanella | Eicopentaenoic acid, docosahexaenoic acid |
| Sphingomonas | |
| Spirulina | |
| Sporolactobacillus | hydroxypropionic acid |
| Streptococcus | |
| Streptomyces | butadiene, avermectin, 1,3-propanediol |
| synechocystis | alkane, alkene, alcohols |
| Thermococcus | |
| Thermotoga | |
| Thiobacillus | |
| Thermosynechoccus | alkane, alkene, alcohols |
| Mannheimia | butadiene |
| Zymomonas | butadiene |
| Lactococcus | butadiene |
| Streptomycetes | rifamycin |
| Actinomycetes | rifamycin |
| Cladosporium | decalactone |
| Micrococcus | citric acid |
| Erwinia | L-phenylalanine |
| Serratia | L-phenylalanine |
| Proteus | L-phenylalanine |
| Kluyvera | L-phenylalanine |

Rewired transcriptional libraries may be probed, as an example, for a number of solutions to problems that currently limit productivity in bacterial fermentation platforms. Bacteriophage contamination represents a serious threat to large scale fermentation. Infection can rapidly proliferate and destroy entire million litre cultures. Rewiring may be used identify strains with, for example, premature lysing effectively destroying cells before virus particles mature. Bacterial strains which are easier to lyse would also be easier to process downstream for protein extraction and purification.

Typically the prokaryotic cell is a Bacillus, or Escherichia. Thus, the prokaryotic cell may be B. subtilis or E. coli or a Synechocystis sp. or a cell as listed in Table 1 above.

When the cell is a prokaryotic cell, for example as indicated above, the regulatory polypeptide may be, for example, a transcription factor (or an enhancer or repressor) or a sigma factor. Information on regulatory polypeptides, for example transcription factors and sigma factors is available, for example from sequence databases. Isalan et al (2008) Nature 452, 840-845 and references cited therein, for example Perez-Rueda & Collado-Vides (2000) The repertoire of DNA-binding transcriptional regulators in Escherichia coli K-12, Nucl Acid Res 28, 1838-1847 and Salgado et al (2006) RegulonDB (version 5.0): Escherichia coli K-12 transcriptional regulatory network, operon organisation, and growth conditions. Nucl Acids Res 34, D394-D397, for example, discuss E. coli regulatory polypeptides. Mutant sigma factors are described in, for example, WO 2007/038564.

When the cell is a prokaryotic cell, for example as indicated above, the regulatory RNA molecule may be a microRNA See, for example, Morita T, Mochizuki Y, Aiba H (2006). “Translational repression is sufficient for gene silencing by bacterial small noncoding RNAs in the absence of mRNA destruction”. Proc Natl Acad Sci USA 103 (13): 4858-63.

The cell may alternatively be a eukaryotic cell, for example a fungal cell, an insect cell, a plant cell or a mammalian cell.

The fungal cell, may be, for example Aspergillus, for example A. fumigatus; or may be a yeast, for example Candida spp; Saccharomyces spp; Pichia spp. For example, the fungal cell may be P. pastoris or S. cerevisiae. Illustrative examples of fungal cells and products which have been produced in those cells (which production, for example, may be increased using methods of the invention) include the following:

TABLE 2
examples of fungal cells and typical products.

| Fungi | Products |
|---|---|
| Ashbya gossypii | riboflavin |
| Aspergilius | itaconic acid, citric acid, aconitic acid, malic acid, cluconic |
| | acid, gamma-linoleic acid, mannitol, enzymes, butadiene, |
| | 1,3-propanediol |
| Aureobasidium | enzymes and siderophores and pullulan |
| Blakslea trispora | beta carotene, lycopene |
| Conidiobolus | dihomo-gamma-linoleic acid |
| Entomophthora | docosahexaenoic acid |
| filobasidium | trehalose |
| Gibberella | givverellic acid |
| Hansenula | enzymes, 1,3-propanediol |
| Kluyveromyces | butadiene, 1,3-propanediol |
| Monascus | monacolin K, cholesterol inhibitors |
| Mortierella | fatty acids (arachidonic acid, omega-3 and 9), (dihomo-)gamma |
| | linoleic acid |
| Mucor | gamma-linoleic acid, 1,3-propanediol |
| Penicillium | cheese making, enzymes, cholesteol inhibitors, antibiotics |
| Pichia | arabitol, enzymes, monacolin K, carotenoids, butadiene, |
| | 1,3-propanediol |
| Pleurotus | trehalose |
| Pythium | 5,8,11,14,17-eicosapentaenoic (omega-3), ARACHIDONIC |
| | ACID |
| Rhizobus | butadiene |
| Rhizopus | butadiene |
| Rhodotorula | hydroxyalkylxanthines, isoflavone aglucone, phenylalanine |
| | analogs, transhydroxy sulfone |
| Saccharomyces | glicerol, ethanol, arabitol, xylitol, carotenoids, butadiene, |
| | 1,3-propanediol |
| Schizosacccharomyces | butadiene, |
| Sporobolomyces | decalactone |
| Thraustochytrium | docosahexaenoic acid |
| Torulopsis | mannitol, 1,3-propanediol |
| Yarrowia lipolytica | lipase, gamma-decalatone, organic acids, Eicosapentaenoic |
| | acid, epoxides and diols, butadiene |
| Zygosaccharomyces | proteins, succinic acid, mailc acid, fumaric acid, B group |
| | vitamins, decalactone, levodione, 1,3-propanediol |
| Candida | 1,3-propanediol |
| Debaryomyces | 1,3-propanediol |

The insect cell may be, for example, Lepidopteran cells, for example, SF9, SF21, High-5, Mimic-SF9. Many suitable cell types and expressed products are known.

The plant cell may be an algal cell; or a monocotyledonous or dicotyledenous plant cell; typically an experimental, crop and/or ornamental plant cell, for example Arabidopsis, maize. Examples of algal cells include Chlamydomonas, Chlorella, Dunalliela, Nannochloropsis, Trichoderma. Products include hydrogen, Short chain volatile hydrocarbon lipids, ascorbic acid, isoprenoids long-chain aldehydes, fatty acids, env control enzymes (cellulases xylanase chitinase). Examples of plant cells include Arabidopsis thaliana, Nicotiana tabacum, Zea mays, soybean Glycine max, Brassica, Helianthus, Gossypium, Medicago, Triticum, Hordeum, Avena, Sorghum, Orya. Many expressed products are known.

Plants experience a number of biotic and abiotic factors that reduce their productivity and slow their growth rate during their life cycle. Plant cells lines and whole plants containing re-wired networks may be screened for improved stress tolerance and productivity under adverse environmental conditions. Other applications for re-wiring in plants include improving the ease and efficiency of transformation and improving the production and specificity of heterologous proteins in selected tissues.

The eukaryotic cell may alternatively be a fish (for example Zebra fish; salmon) or bird (for example chicken or other domesticated bird).

The eukaryotic cell may be a mammalian cell. The cell may, for example, be a human, mouse, rat, rabbit, bovine or dog (or, for example, any other wild, livestock/domesticated animal) cell. The cell may, for example, be a stable cell line cell, or a primary cell, adherent or suspension cell. As examples, the cell may be a HeLa cell line cell or a mouse primary cell, or a CHO (Chinese Hamster Ovary) cell Many product types are known, as discussed further below, for example many types of polypeptide. Mammalian cell lines are being used extensively in the production of therapeutic proteins for research in clinical trials, mainly due to their ability to produce near identical, functional human proteins. However, these cells are very susceptible to contamination and toxins that build up during fermentation processes. Re-wiring could be used to identify cells lines with improved growth rates capable of more effectively outcompeting contamination. Transcriptional re-wiring could also be used to identify cell lines with improved performance under stress or toxin conditions experienced during protein fermentation.

When the cell is a eukaryotic cell, the regulatory polypeptide may be a transcription factor, for example.

Examples of suitable groups of transcription factor, for example, include the following (for example in relation to P. pastoris):

Transcription from RNA polymerase II promoter

Response to chemical stimulus

Signalling

Response to osmitic stress

Chromatin organisation

Regulation of transport

Pseudohyphal growth

Protein complex biogenesis

Cellular ion homeostasis

Transmembrane transport

Response to DNA damage stimulus

Protein modification by small protein conjugation or removal

Mitotic cell cycle

Response to oxidative stress

Response to heat

Protein phosphorylation

Examples of regulatory polypeptides include:

Sir2

N cat rep (Nitrogen catabolite Repressor; 8198117)

Proteosome TF

Glucose Rep TF

26S proteasome Reg

CCHC zinc-finger (for example 8198201)

RNA Pol III neg Reg

Heat Shock TF (8200994)

mRNA Transport Reg

Trimeric Heat Shock TF

18S rRNA maturation factor (8198708)

In particular, the following regulatory polypeptides may be used, for example in relation to Pichia spp. Homologues, for example, may be useful when using the invention in other eukaryotic cells. Methods for identifying homologues will be well known to those skilled in the art, and include sequence comparisons, which are also well known to those skilled in the art. See, for example, US 2008/0085535. The level of sequence identity needed to identify a gene as a homolog will depend on how close phylogenetically the source organisms are. Typically the level of sequence identity may be at least 50%, for example at least 60%, 70%, 80% or 90%. The reference to any particular regulatory polypeptide includes reference to a mutant polypeptide in which one or more amino acids is changed: the level of identity with the wild type polypeptide is typically at least 80%, 90%, 95% or 98%.

TABLE 3
regulatory polypeptides

| GeneID: | Description: |
|---|---|
| 8196484 | DNA-binding protein involved in either activation or repression of |
| | transcription |
| 8196642 | bZIP transcription factor (ATF/CREB1 homolog) that regulates the |
| | unfolded protein response |
| 8196675 | Negative regulator of the glucose-sensing signal transduction pathway |
| 8197110 | Regulatory protein MIG1 |
| 8197136 | Subunit of the CCR4-NOT complex, which is a global transcriptional |
| | regulator with roles in transcription |
| 8197166 | Positive regulator of genes in multiple nitrogen degradation pathways |
| 8197205 | Non-ATPase regulatory subunit of the 26S proteasome |
| 8197237 | Protein involved in negative regulation of transcription of iron regulon |
| 8197559 | Multistep regulator of cAMP-PKA signalling |
| 8197661 | hypothetical protein |
| 8197765 | Member of silencing information regulator 2 (Sir2) family of NAD(+)- |
| | dependent protein deacetylases |
| 8197788 | Transcriptional activator |
| 8197813 | Subunit of the SAGA transcriptional regulatory complex but not present |
| | in SAGA-like complex SLIK/SAL |
| 8197959 | negative regulator of RNA polymerase III |
| 8197996 | PIK-related protein kinase and rapamycin target |
| 8198145 | Trimeric heat shock transcription factor, activates multiple genes in |
| | response to stresses |
| 8198152 | Nitrogen catabolite repression transcriptional regulator that acts by |
| | inhibition of GLN3 transcription |
| 8198177 | Ubiquitin-conjugating enzyme involved in the error-free DNA |
| | postreplication repair pathway |
| 8198201 | Protein with seven cysteine-rich CCHC zinc-finger motifs, similar to |
| | human CNBP |
| 8198232 | Protein kinase involved in the response to oxidative and osmotic stress |
| 8198277 | Transcription factor that activates transcription of genes involved in |
| | stress response |
| 8198289 | Transcriptional regulator involved in glucose repression of Gal4p- |
| | regulated genes |
| 8198341 | Transcriptional activator related to Msn2p |
| 8198430 | Transcription factor involved in regulation of cell cycle progression from |
| | G1 to S phase |
| 8198510 | Sterol regulatory element binding protein, induces transcription of sterol |
| | transport and biosynthetic enzymes |
| 8198926 | Subunit of TORC1, a rapamycin-sensitive complex involved in growth |
| | control |
| 8198945 | Protein involved in negative regulation of transcription of iron regulon |
| 8199654 | Ser/Thr kinase involved in transcription and stress response |
| 8200275 | Copper-sensing transcription factor |
| 8200338 | Cytoplasmic response regulator, part of a two-component signal |
| | transducer that mediates osmosensing |
| 8200389 | Transcription factor that stimulates expression of proteasome genes |
| 8200775 | Transcription factor involved in glucose repression |
| 8200866 | Basic leucine zipper (bZIP) transcription factor required for oxidative |
| | stress tolerance |
| 8200877 | Forkhead family transcription factor with a major role in the expression |
| | of G2/M phase genes |
| 8200938 | Essential nuclear protein with a possible role in the osmoregulatory |
| | glycerol response |
| 8200968 | pH-response regulator protein |
| 8200994 | Heat shock protein with a zinc finger motif |
| 8201004 | mRNA transport regulator, essential nuclear protein |
| 8201078 | Transcription factor that controls expression of many ribosome |
| | biogenesis genes |
| 8201128 | Protein kinase involved in the response to oxidative and osmotic stress |
| 8201180 | Putative transcription factor involved in regulating the response to |
| | osmotic stress |
| 8201433 | Carbon source-responsive zinc-finger transcription factor |

Other regulatory products may include tRNA genes.

For example, it may be appropriate to express tRNA genes, for example alongside other constructs, particularly when seeking to express a desired product that has a skewed codon usage relative to the host cell. For example, this may apply in relation to silk proteins, as discussed further in the Examples. As an example, when expressing a silk protein in P. pastoris it may be desirable to express tRNA genes under control of the AOX1 promoter; optionally also alongside a construct containing the Cat A promoter (8198267), as described in the Examples.

Examples of suitable groups of promoter regions, for example, include the following (for example in relation to P. pastoris):

Transcription from RNA polymerase II promoter

Response to chemical stimulus

Transmembrane transport

Signalling

Response to oxidative stress

Response to osmotic stress

Cellular amino acid metabolic process

Chromatin organisation

Pseudohyphal growth

Ion transport

Cellular ion homeostasis

Response to DNA damage stimulus

Regulation of transport

Protein targeting

Protein phosphorylation

Protein complex biogenesis

Carbohydrate metabolic process

Protein modification by small protein conjugation or removal

Mitotic cell cycle

Regulation of cell cycle

Response to heat

Examples of promoter regions include:

Catalase A (8198267)

Glucose Rep TF

Cell Cycle TF

AOX

Heat Shock TF

Stress Resp TF

MSn2p related

S-phase Ind Earn

Cytoplasmic resp reg

Proline permease (8199273)

Lysine permease (8196606)

The Catalase A (8198267) promoter may, for example, be effective in the absence of a separate regulatory product-encoding region.

In particular, the following promoter regions may be used, for example in relation to Pichia spp. Homologues, for example, may be useful when using the invention in other eukaryotic cells.

TABLE 4
promoter regions

| GeneID: | Description |
|---|---|
| 8196484 | DNA-binding protein involved in either activation or repression of |
| | transcription |
| 8196642 | bZIP transcription factor (ATF/CREB1 homolog) that regulates the unfolded |
| | protein response |
| 8196675 | Negative regulator of the glucose-sensing signal transduction pathway |
| 8197110 | Regulatory protein MIG1 |
| 8197136 | Subunit of the CCR4-NOT complex, which is a global transcriptional |
| | regulator with roles in transcription |
| 8197166 | Positive regulator of genes in multiple nitrogen degradation pathways |
| 8197205 | Non-ATPase regulatory subunit of the 26S proteasome |
| 8197237 | Protein involved in negative regulation of transcription of iron regulon |
| 8197559 | Multistep regulator of cAMP-PKA signalling |
| 8197661 | hypothetical protein |
| 8197765 | Member of silencing information regulator 2 (Sir2) family of NAD(+)- |
| | dependent protein deacetylases |
| 8197788 | Transcriptional activator |
| 8197813 | Subunit of the SAGA transcriptional regulatory complex but not present in |
| | SAGA-like complex SLIK/SAL |
| 8197959 | negative regulator of RNA polymerase III |
| 8197996 | PIK-related protein kinase and rapamycin target |
| 8198145 | Trimeric heat shock transcription factor, activates multiple genes in |
| | response to stresses |
| 8198152 | Nitrogen catabolite repression transcriptional regulator that acts by |
| | inhibition of GLN3 transcription |
| 8198177 | Ubiquitin-conjugating enzyme involved in the error-free DNA |
| | postreplication repair pathway |
| 8198201 | Protein with seven cysteine-rich CCHC zinc-finger motifs, similar to human |
| | CNBP |
| 8198232 | Protein kinase involved in the response to oxidative and osmotic stress |
| 8198277 | Transcription factor that activates transcription of genes involved in stress |
| | response |
| 8198289 | Transcriptional regulator involved in glucose repression of Gal4p-regulated |
| | genes |
| 8198341 | Transcriptional activator related to Msn2p |
| 8198430 | Transcription factor involved in regulation of cell cycle progression from G1 |
| | to S phase |
| 8198510 | Sterol regulatory element binding protein, induces transcription of sterol |
| | transport and biosynthetic enzymes |
| 8198926 | Subunit of TORC1, a rapamycin-sensitive complex involved in growth control |
| 8198945 | Protein involved in negative regulation of transcription of iron regulon |
| 8199654 | Ser/Thr kinase involved in transcription and stress response |
| 8200275 | Copper-sensing transcription factor |
| 8200338 | Cytoplasmic response regulator, part of a two-component signal transducer |
| | that mediates osmosensing |
| 8200389 | Transcription factor that stimulates expression of proteasome genes |
| 8200775 | Transcription factor involved in glucose repression |
| 8200866 | Basic leucine zipper (bZIP) transcription factor required for oxidative stress |
| | tolerance |
| 8200877 | Forkhead family transcription factor with a major role in the expression of |
| | G2/M phase genes |
| 8200938 | Essential nuclear protein with a possible role in the osmoregulatory glycerol |
| | response |
| 8200968 | pH-response regulator protein |
| 8200994 | Heat shock protein with a zinc finger motif |
| 8201004 | mRNA transport regulator, essential nuclear protein |
| 8201078 | Transcription factor that controls expression of many ribosome biogenesis |
| | genes |
| 8201128 | Protein kinase involved in the response to oxidative and osmotic stress |
| 8201180 | Putative transcription factor involved in regulating the response to osmotic |
| | stress |
| 8201433 | Carbon source-responsive zinc-finger transcription factor |
| 8201223 | Alcohol oxidase |
| 8197797 | Peroxisomal biogenesis factor 8 |
| 8198404 | Ribosomal protein 59 of small subunit, required for ribosome assembly and |
| | 20S pre-rRNA processing |
| 8200362 | Ferrioxamine B transporter |
| 8198915 | Non-essential subunit of Sec63 complex (Sec63p, Sec62p, Sec66p and |
| | Sec72p) |
| 8198174 | Plasma membrane transporter for both urea and polyamines, expression is |
| | highly sensitive to nitrogen |
| 8197884 | Ammonium permease involved in regulation of pseudohyphal growth |
| 8199755 | Cytosolic and mitochondrial glutathione oxidoreductase |
| 8198267 | Catalase A, breaks down hydrogen peroxide in the peroxisomal matrix |
| | formed by acyl-CoA oxidase (Pox1) |
| 8198279 | Thioredoxin peroxidase, acts as both a ribosome-associated and free |
| | cytoplasmic antioxidant |
| 8196981 | Member of a stationary phase-induced gene family |
| 8199313 | Plasma membrane transporter for both urea and polyamines, expression is |
| | highly sensitive to nitrogen |
| 8200226 | High affinity sulfate permease |
| 8199939 | Major of three pyruvate decarboxylase isozymes |
| 8199846 | Fumarase, converts fumaric acid to L-malic acid in the TCA cycle |
| 8200841 | NADPH-dependent medium chain alcohol dehydrogenase with broad |
| | substrate specificity |
| 8197066 | General amino acid permease |
| 8197738 | Aromatic aminotransferase I, expression is regulated by general control of |
| | amino acid biosynthesis |
| 8199681 | Glutamate decarboxylase, converts glutamate into gamma-aminobutyric |
| | acid (GABA) |
| 8197683 | Ammonium permease |
| 8196441 | Permease of basic amino acids in the vacuolar membrane |
| 8199273 | Proline permease, required for high-affinity transport of proline |
| 8196606 | Lysine permease |
| 8199308 | High affinity methionine permease, integral membrane protein with 13 |
| | putative membrane-spanning regi |
| 8198785 | Sulfiredoxin, contributes to oxidative stress resistance by reducing cysteine- |
| | sulfinic acid groups i |

When the cell is an eukaryotic cell, the regulatory RNA molecule may alternatively be a microRNA. See, for example Bartel, D (2004). “MicroRNAsGenomics, Biogenesis, Mechanism, and Function”. Cell 116 (2): 281-297 and He, Lin; Hannon, Gregory J. (2004). “MicroRNAs: small RNAs with a big role in gene regulation”. Nature Reviews Genetics 5 (7): 522-531

Promoter and regulatory product selection for the plurality of distinct nucleic acid molecule species (libraries) may be carried out by interrogating public transcriptomics studies that investigate specific or similar conditions for which a new phenotype is required. For instance identifying and selecting promoter and ORF of transcriptional regulators that exhibit transcriptional variability over different plant stresses. Similarly libraries for investigating bacterial lysis may be assembled based on transcriptomic information on natural processes of cell division, growth and pathogen attack to understand how cell lysis can be manipulated. For mammalian cell lines, data that captures transcriptomic variation during adverse fermentation conditions can help identify transcriptional regulators whose manipulation will allow the evolution of new desired phenotypic behaviour. The construction of putative regulatory networks from these transcriptome data could help highlight key network drivers based on a regulator's connectivity or influence on particular parts of the network known to influence the phenotypic trait under investigation. Selection can be further refined by identifying regulators with supporting experimental evidence from knockdown or overexpression studies. Transcription factors that are considered to regulate a moderate number (ie more than just one or two, but less than about 20, 15 or 10; typically considered not of be a “master regulator”) may be particularly useful to include. Knowledge gained through preliminary library screens could be used to iteratively inform subsequent library construction to improve phenotype discovery efficiency.

Thus, the library may be designed by a method in which relevant promoter regions and genes are identified by means of computational approaches. Genome annotation tools may be used to identify regions of interest according to
Similarity to regions of known function
Previous scientific knowledge of the region.

As will be apparent to the skilled person, individual or a combination of computational methods may be used to identify regions of interest according to their biological function. As examples, the computational methods may be ones which infer biological function from one or more of sequencing, transcriptomics, metabolomics, immunoprecipitation (or other interaction/binding) data. Relevant data will already be in the public domain. Further data may, for example, be produced by means of DNA sequencing, for example. Regions of interest may be identified by sequence homology to known biologically relevant DNA sequences and/or biophysical properties. See WO 2007/072214, for example, for discussion of identification of related sequences. The data may alternatively be produced by means of, for example, DNA microarrays, RNA microarrays, SAGE, SuperSAGE, RNA-Seq or RNA-seq. Regions of interest may be identified by patterns of gene expression, such as but not limited to first time of differential expression or overall differential expression. The data may also be produced by means of protein microarrays, LC-MS, one-hybrid, two-hybrid, three-hybrid or phage display. Regions of interest may be identified by the relevance of detected protein-protein interactions towards regulatory mechanisms.

The data may alternatively be produced by means of Chip-chip arrays, RIP-Chip, as well known to those skilled in the art. Regions of interest may be identified by the analysis of binding of transcription factors, enhancers, repressors and other regulatory elements to DNA fragments. The data may be produced by means of CLIP-Seq (a method for identifying the binding sites of cellular RNA-binding proteins (RBPs) using UV light to cross-link RNA to RBPs without the incorporation of photoactivatable groups into RNA) or PAR-CLIP (Photoactivatable-Ribonucleoside-Enhanced Crosslinking and Immunoprecipitation). Thus, regions of interest may be identified by the existence of regulation by protein binding to RNA molecules.

The data may alternatively be produced by means of GC-MS or LC-MS, as well known to those skilled in the art. Regions of interest may be identified by mapping levels of metabolites to genes responsible for their production and known regulators.

Regions of interest may be selected by the inference of their behaviour in simulated networks, for example
a) using models generated with information collected from individual or combinations of methods such as sequencing, transcriptomics.

It will be appreciated, however, that it is not considered essential for the plurality of distinct nucleic acid molecule species which is introduced into the population of cells to have been designed based on extensive information; or “tailored” to a particular characteristic or product. Preparation of a tailored or focused library may be useful in some circumstances and may reduce the number of cells that have to be screened in order to identify cells with increased production; but a library with no or less stringent pre-selection of components may also be useful, for example in enabling progress to be made when little is known about what may be limiting factors, for example in a cell type that has not previously been used or studied extensively, or in allowing a broader range of options to be considered. As is apparent from the examples, the same library was used successfully in generating cell lines with enhanced production of polypeptides and enhanced production of alkanes, for example.

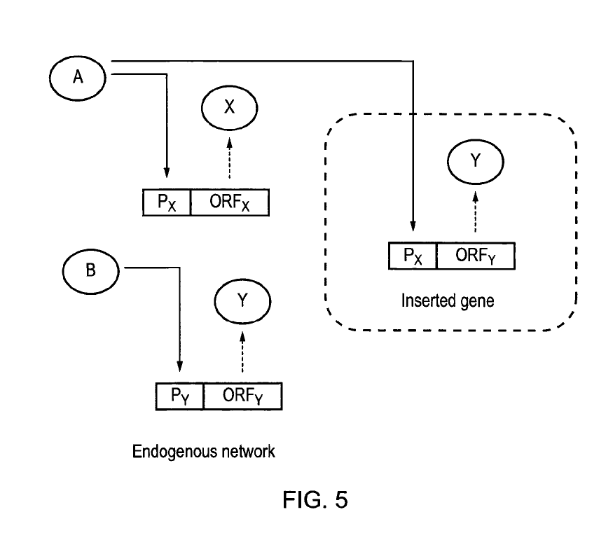

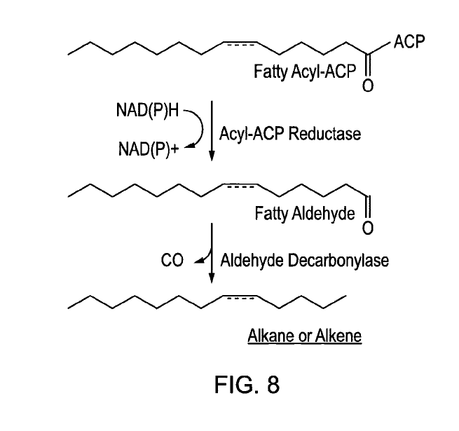

Examples of sources of information on regulatory products and promoters include, for example, WO 03/062943 and references therein, for example in relation to E. coli and S. cerevisiae, for example as discussed on pages 21 to 30 and references and Figures (for example FIGS. 5 and 8) cited therein; WO 00/32748 (see, for example, Tables 1 and 2 which indicate promoters

As noted above, the desired product may be, for example, an industrial enzyme, a therapeutic protein, a membrane protein, a functional protein material, a fatty acid, an alcohol, a terpenoid, a bioplastic, a pigment, or an alkaloid drug. Typically the desired product is a heterologous product.

As examples, the methods of the invention may be applied to:

Ia. High Value Proteins.

Recombinant human growth factors (ie epidermal growth factor, EGF; fibroblast growth factor, FGF; brain derived neurotrophic factor, BDNF), for example, are difficult to produce and have a high market value ($1000s per milligram). Growth factors are used in research and mammalian cell culture applications and are currently produced by E. coli or Pichia pastoris. The rewiring platform may be used to improve expression and secretion of growth factors in, for example, P. pastoris.

1b. Nutritional Supplements.

The rewiring platform may be used to improve the fermentative production of, for example, the high value supplement lycopene. Lycopene is used as a nutritional supplement and food colouring and currently produced by extraction from tomatoes or natural algae sources. Attempts at fermentative production (ie growing natural or recombinant lycopene-producing microbes in culture) have been plagued by low productivities (on the order of mg per litre) and are not commercially viable. The current price of lycopene is approximately $6000 per kilogram. We estimate that fermentative yields should be greater than 1-5 g/L.

Other Bioproducts.

An increasing number of pharmaceutical, industrial, and chemical products are manufactured using microbial organisms. However, selection and optimization of ‘production strains’ is largely a trial-and-error process, requiring significant time and resource investments to increase product yields or develop strains for new products. Examples of products and companies in each space is listed below:

Biologics and Therapeutics.

Encompasses protein-based products such as therapeutic and diagnostic antibodies, growth factors, and other recombinant proteins. Of the recombinant proteins on the market, 40% are produced by bacteria (almost all by E. coli), 15% by yeasts, and 45% by mammalian cells. ‘Blockbuster’ drug production is dominated by E. coli, S. cerevisiea, and CHO cells.

Industrial Enzymes.

These are typically lower cost but high volume products. Amylases, proteases, and glucanases, for example, are used in food and beverage processing, cellulases and ligninases used in paper manufacturing, and proteases used in detergents.

Biochemicals.

Bulk products such as amino acids, specialty products include food additives, nutraceuticals, and vitamins. Could also include metabolically engineered drug pathways.

Green and Clean Tech.

Products include renewable fuels and chemicals (ethanol and other alcohols, alkanes) from biomass, bio-based materials.

Research Suppliers.

Products may be, for example, restriction enzymes, polymerases, ligases; also could encompass strains specialized in carrying high copy plasmids, expression of toxic proteins, or enhanced transformation and/or recombination potential.

Environmental Applications.

Applications where microbes are being explored in resource extraction, recovery, and waste management. These include biomining, microbially enhanced oil recovery, and anaerobic digestion of municipal and agricultural wastes to biogas.

The desired product may be, for example, a growth factor, a cellulase, an amylase, a lipase, an antibody or an antibody fragment, an immunotoxin, a GPCR, a spider silk, an adhesive protein, an alkane, an alkene, a FAME, methanol, ethanol, isobutanol, a mevalonate-derived fuel, a mevalonate-derived drug, a polyhydroxybutyrate, a polyhydroxybutanoate, a lactam, or lycopene.

In an example, the cell is from Pichia ssp or P. pastoris and the desired product is a growth factor; a human opioid receptor, insulin, an antibody fragment, a spider silk polypeptide or an immunotoxin therapeutic (for example as discussed further in Example 1).

Typically the desired product is not Green Fluorescent Protein (GFP). It may be useful for the desired product to be tagged with a GFP or similar moiety whilst performing the method of identifying a cell with increased production of a desired product.

In an example, the cell is P. pastoris, the desired product is silk protein, the first region comprises a promoter from a P. pastoris gene encoding lysine permease protein 8196606, and the second region comprises a nucleic acid sequence that encodes P. pastoris protein 8198201.

In a further example the cell is P. pastoris, the desired product is an scFv antibody, the first region comprises a promoter from a P. pastoris gene encoding protein 8197996, and the second region comprises a nucleic acid sequence that encodes P. pastoris protein 8200775.

In a further example the cell is P. pastoris, the desired product is insulin, the first region comprises a promoter from a P. pastoris gene encoding protein 8198945, and the second region comprises a nucleic acid sequence that encodes P. pastoris protein 8198341.

Further examples will be apparent from the Examples.

A further aspect of the invention provides a cell produced by, identified by, or isolated by any of the preceding methods of the invention, or a descendant thereof, with increased production of the desired product.

A further aspect of the invention provides a method for increasing the production of a desired product, the method comprising:
producing, identifying, or isolating, by the method of any of the preceding aspects of the invention, cells with increased production of the desired product;
culturing the cells, or descendants thereof with increased production of the desired product, under conditions in which the cells express the desired product; and
recovering the increased amount of the desired product from the cells or the cell culture.

The method may further comprise isolating, concentrating, purifying and/or formulating the increased amount of the desired product. Appropriate methods will be well known to those skilled in the art for particular desired products.

A further aspect of the invention provides a method for bioremediation of a waste product, the method comprising:
providing cells produced by, identified by, or isolated by any of the appropriate methods of the invention, or descendants thereof, with increased production of a desired product that metabolises the waste product; and
exposing the cells to the selected waste product, thereby providing bioremediation of the selected waste product.

As will be known to those skilled in the art, the waste product may be a metal;

hydrocarbon oil; or municipal, industrial (for example paper of fibre processing) or agricultural waste

A further aspect of the invention provides a collection of cells that produce a desired product, wherein the cells in the collection comprise a plurality of distinct nucleic acid molecule species,
wherein each nucleic acid molecule comprises a first region which is operably-linked to a second region, wherein the first region comprises a promoter region from a first gene, and the second region comprises a nucleic acid sequence from a second gene that is capable of expressing a regulatory product under control of the promoter region, and
wherein the desired product is not GFP.

The desired product may (or may not) be labelled with a GFP moiety, but the GFP is not itself the desired product.

The plurality of distinct nucleic acid molecule species may comprise a plurality of combinations of multiple first and multiple second regions. The nucleic acid molecules may be integrated into the genome of the cells that produce the desired product or may be maintained extra-chromosomally in the cells that produce the desired product.

Preferences are otherwise as set out for preceding aspects of the invention. The plurality of distinct nucleic acid molecule species typically, for example, has been selected or designed as indicated above.

A further aspect of the invention provides a kit of parts comprising:
a population of cells that produce a desired product; and a plurality of distinct nucleic acid molecule species, wherein each nucleic acid molecule comprises a first region which is operably-linked to a second region, wherein
   the first region comprises a promoter region from a first gene, and
   the second region comprises a nucleic acid sequence from a second gene that is capable of expressing a regulatory product under control of the promoter region in the cells that produce the desired product.

The plurality of distinct nucleic acid molecule species may comprise a plurality of combinations of multiple first and multiple second regions, for example as noted above in relation to the first aspect of the invention. The plurality of distinct nucleic acid molecule species typically, for example, has been selected or designed as indicated above.

Typically the desired product is not encoded by the nucleic acid molecule species.

The plurality of nucleic acid molecule species may be contained in expression vectors, (as is also the case for preceding aspects of the invention). The plurality of nucleic acid molecule species may be contained in integration vectors that are capable of integration into the genome of the cells that produce the desired product; or may be contained in episomal vectors that are capable of extra-chromosomal maintenance in the cells that produce the desired product. Examples of both types of vector will be well known to those skilled in the art. See, for example, US 2008/0085535 and references cited therein.

Preferences are otherwise as set out for preceding aspects of the invention.

The invention is now described in more detail by reference to the following, non-limiting, Figures and Examples.

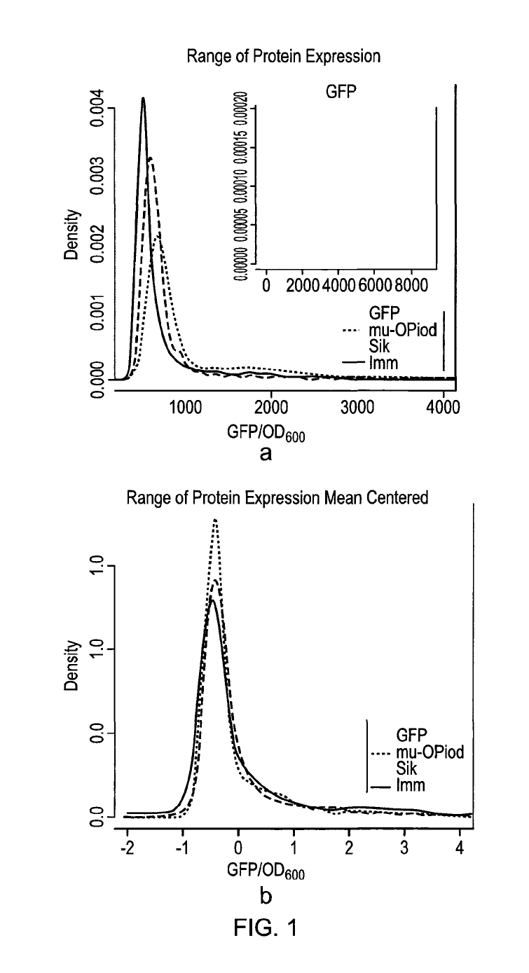

FIG. 1. Primary Screen: a Raw expression distribution of four libraries. b Mean centring and standardising library data brings all the means to 0 and standard deviations to 1 allowing objective selection of outliers with enhanced expression two standard deviations greater than the mean of the library. GFP: endogenously expressed GFP, mu-Opiod: membrane localised opiod receptor with a C-terminal GFP fusion, Silk: secreted spider silk with a C-terminal GFP fusion and Imm:

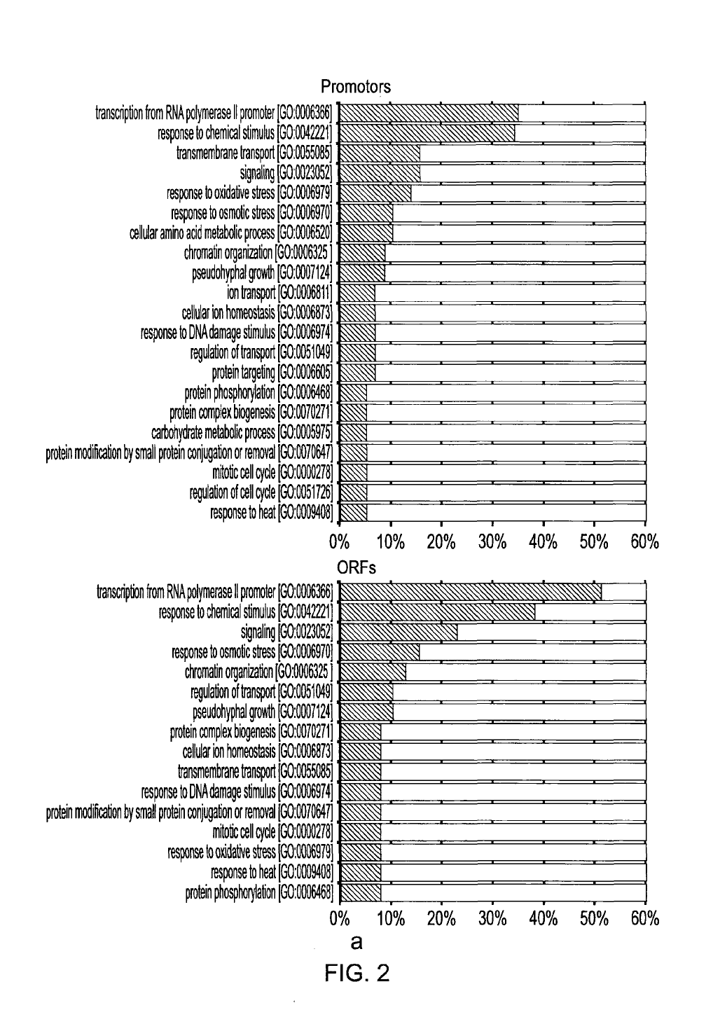

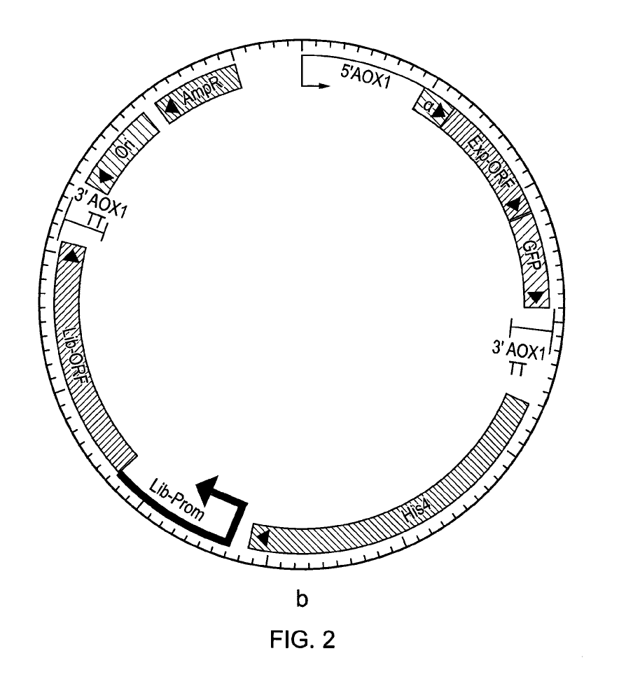

FIG. 2: Library: A library was designed to randomly combine promoters and open reading frames (ORFs) from different endogenous genes involved in a number of biological processes relevant to the efficiency of heterologous protein expression. a Enrichment analysis for the GO biological process in the 67 promoters and 43 ORFs selected for library assembly. b Plasmid design for screening the library in Pichia pastoris. Promoters were fused upstream of ORFs and the AOX1 terminator sequence was placed downstream of ORFs.

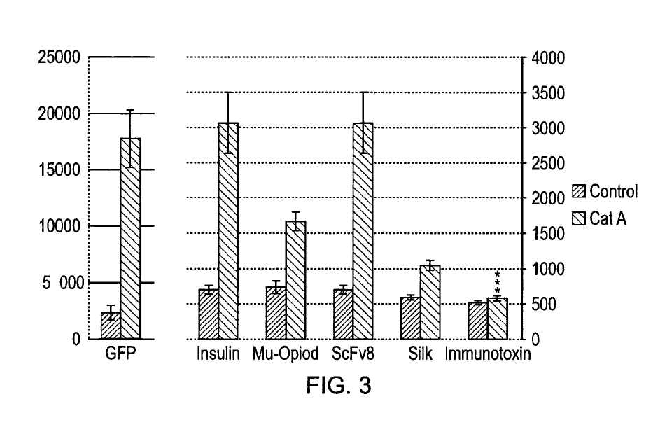

FIG. 3. 18S Mat: Enhanced expression of six different heterologous proteins mediated by the presence of a duplicated promoter region of Catalase A. Significance of change in expression was assessed using a ttest. p-value 0.00005<***<0.0001. All other comparisons between control and CatA promoter obtained p-value<1 −e⁶.

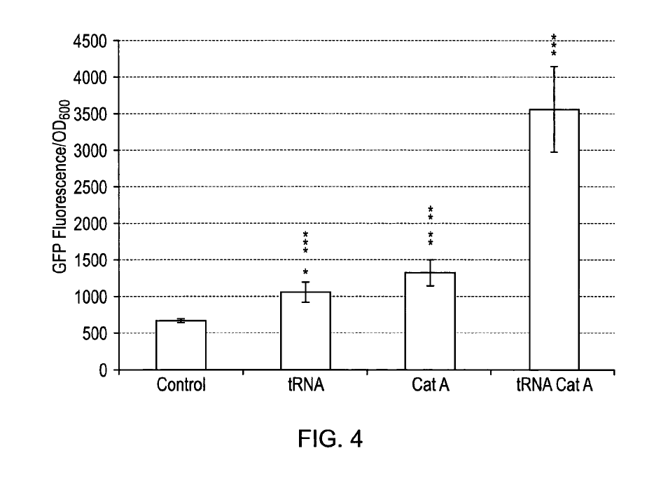

FIG. 4. Silk Production: Mean OD₆₀₀ normalised expression levels for 40 colonies of a control strain (silk only no rewired components), tRNA: silk expressed in conjunction with the Gly-Ala-Gly-Ala-Gly tRNA construct, CatA: truncated Catalyse A promoter. Error bars show standard error. Ttests performed to compare lines to control (black asterisks) and to tRNA Cat A double rewiring line (red asterisks). p-value: *<0.05, **<0.001 and ***<0.0001

FIG. 5. Network rewiring

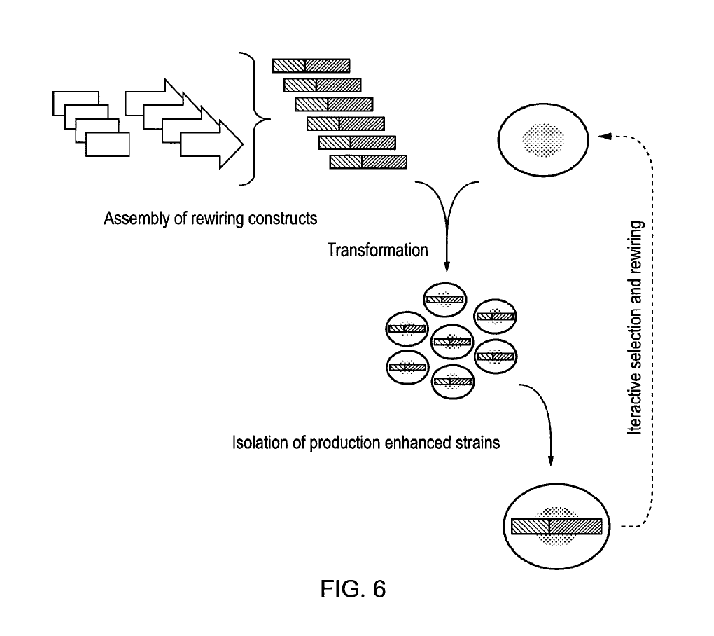

FIG. 6. Assembly of rewiring constructs

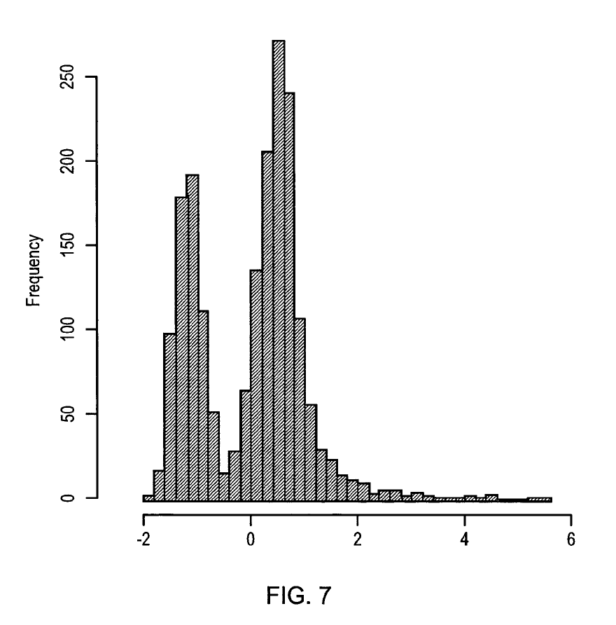

FIG. 7. Sample of data from rewiring of P. pastoris expressing GFP under control of the highly methanol inducible AOX promoter.

FIG. 8. Alkane or Alkene production

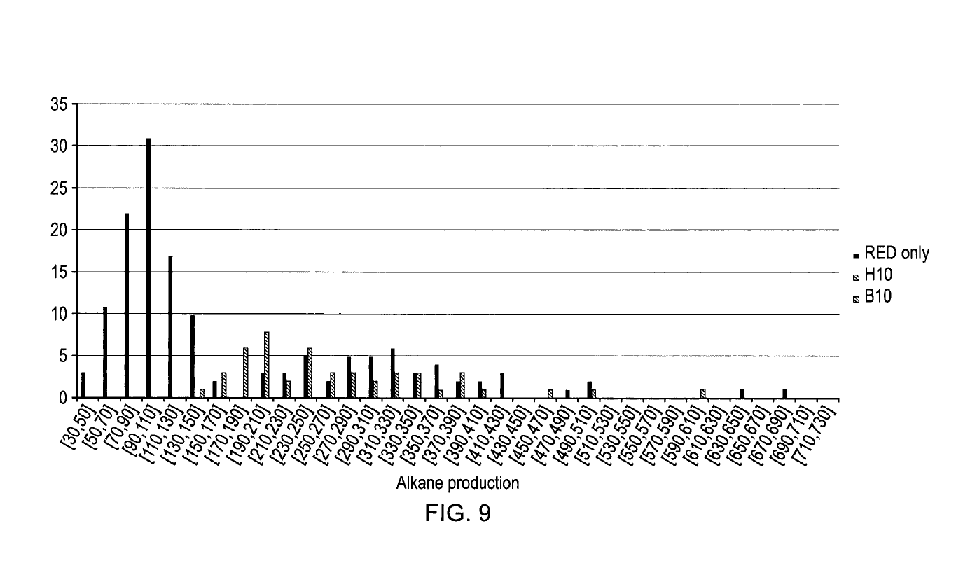

FIG. 9. Alkane production following an additional round of library generation and screening.

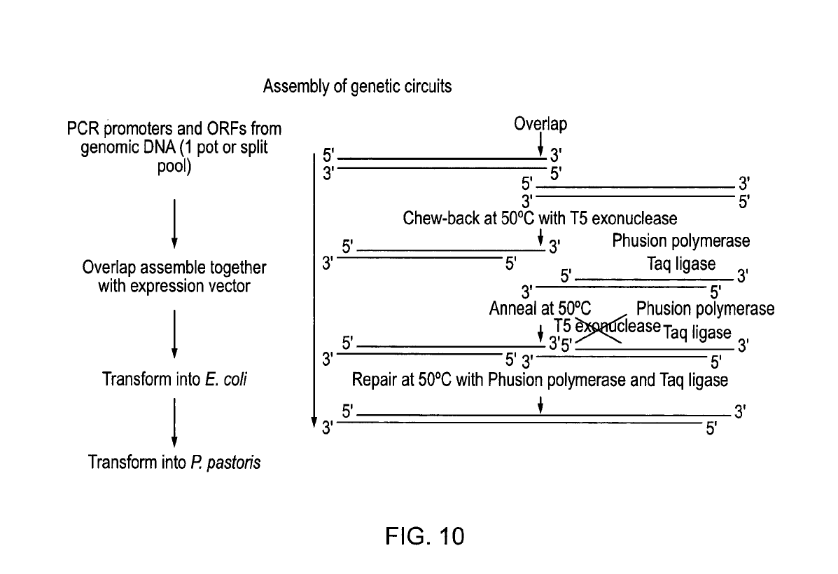

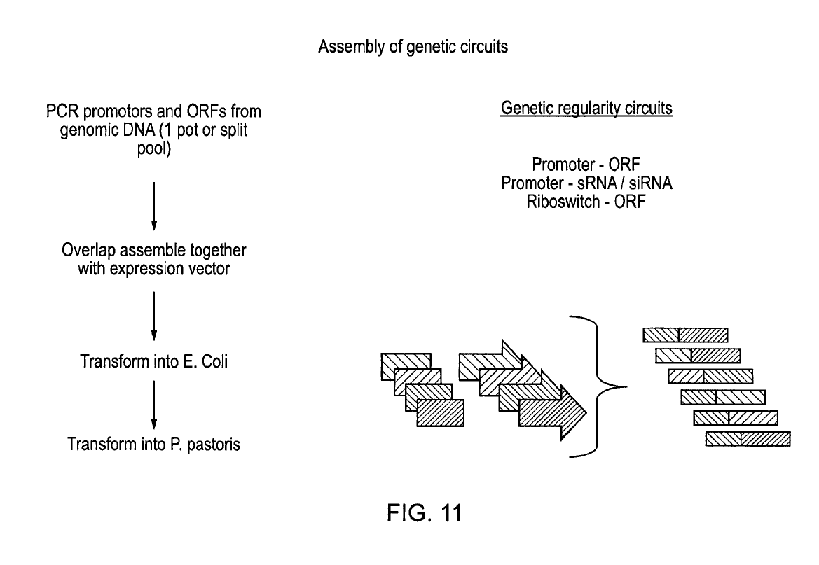

FIGS. 10 and 11. Examples of assembly of genetic circuits

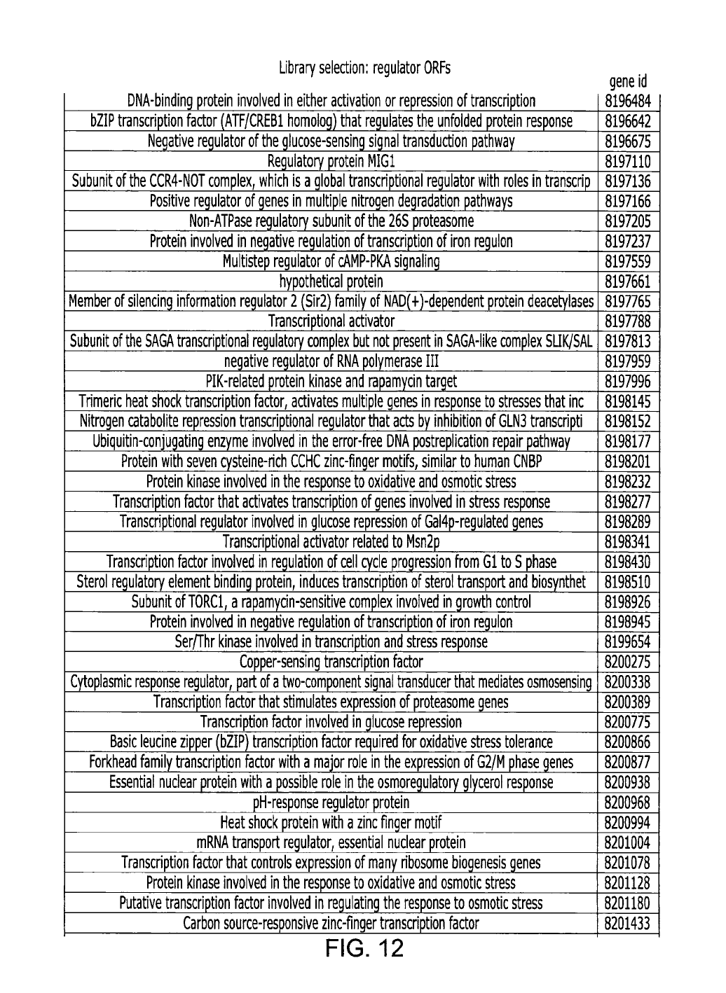

FIG. 12. Examples of regulator ORFs

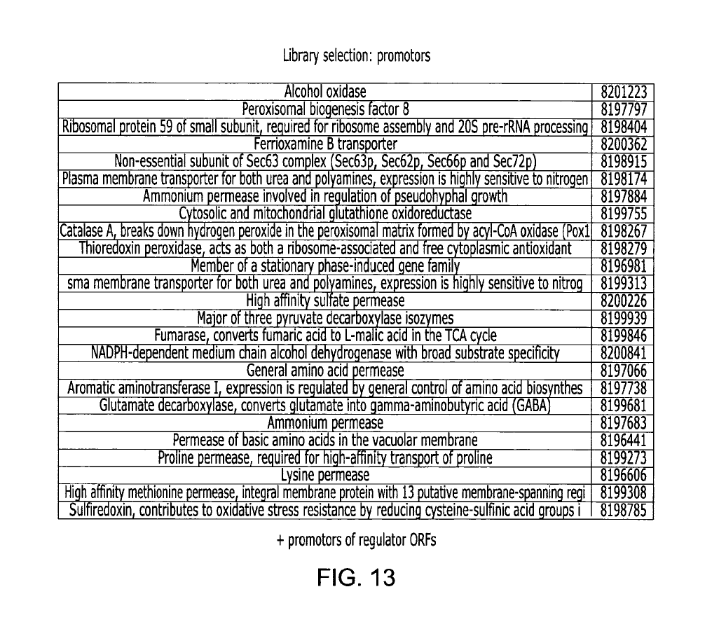

FIG. 13. Examples of promoters

FIG. 14. Examples of product detection strategies

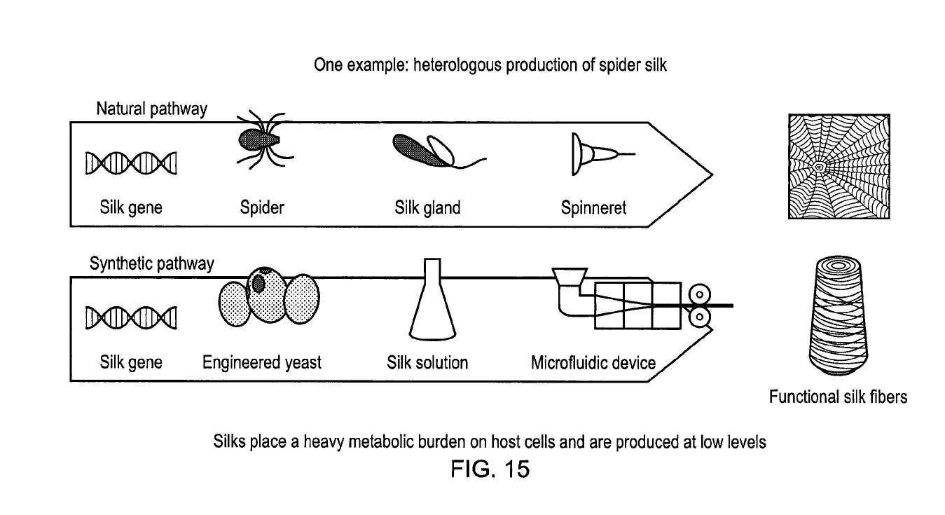

FIG. 15. Heterologous production of spider silk

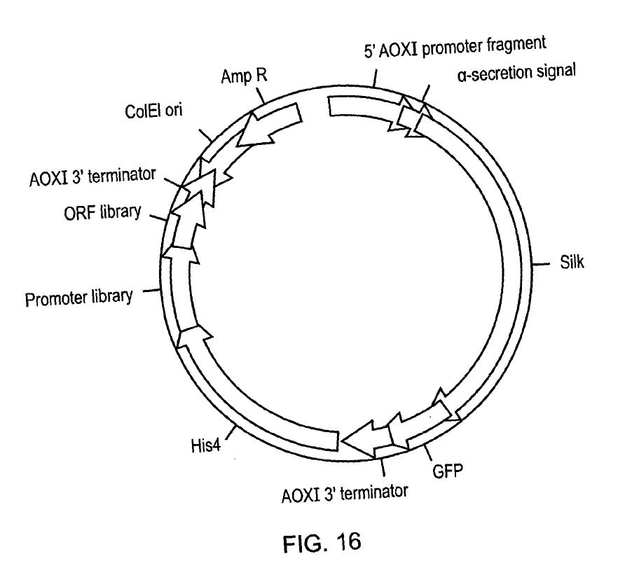

FIG. 16. Expression plasmid for silk production

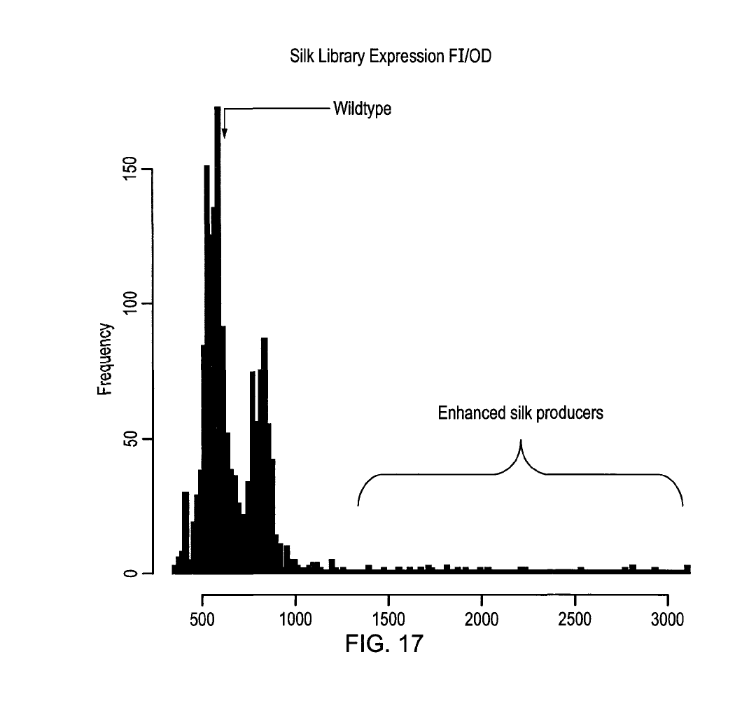

FIG. 17. Selection of enhanced silk producers

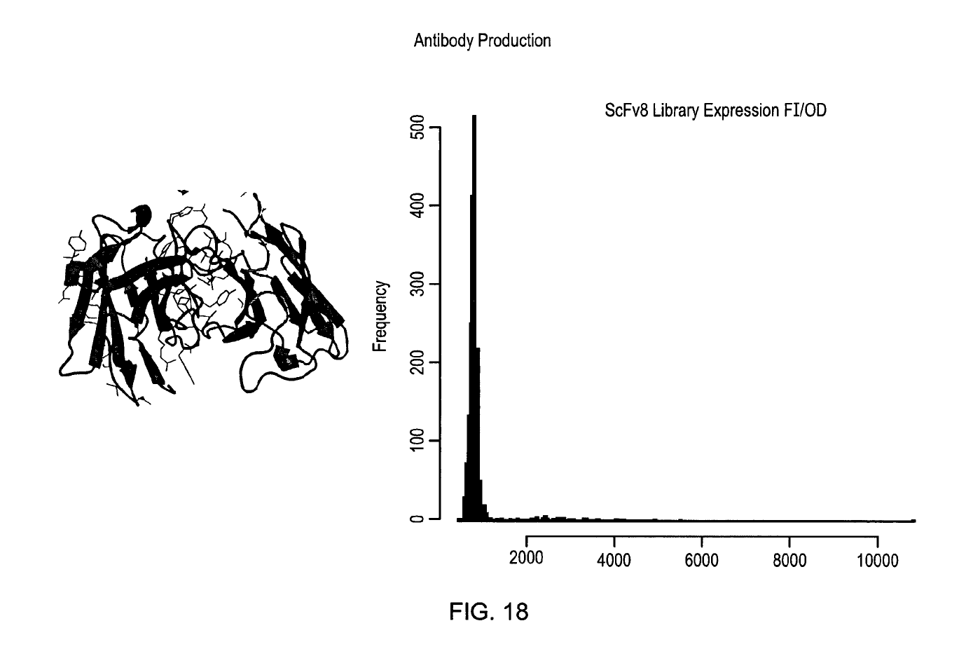

FIG. 18. Selection of enhanced antibody fragment producers

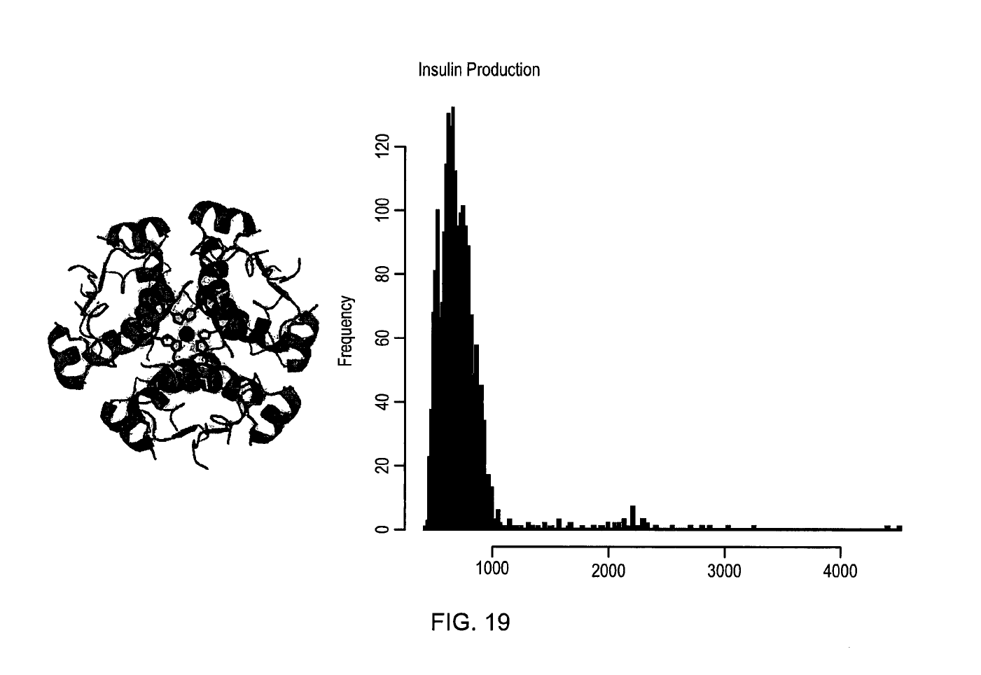

FIG. 19. Selection of enhanced insulin producers

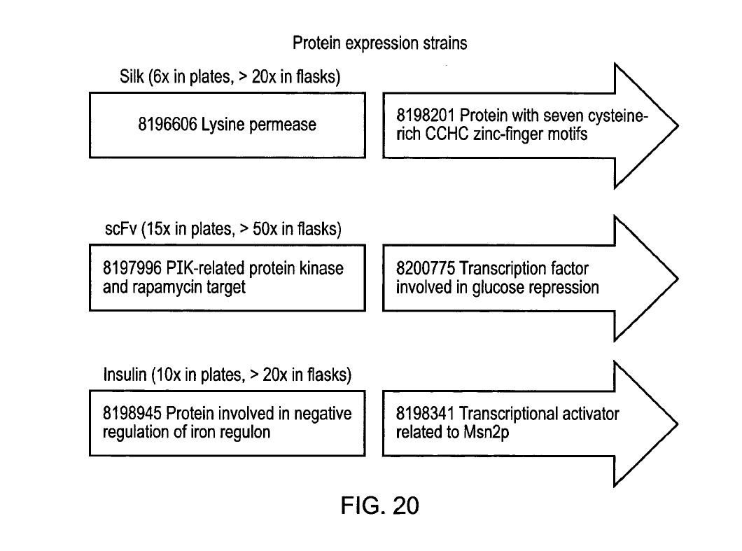

FIG. 20. Enhanced expression strains

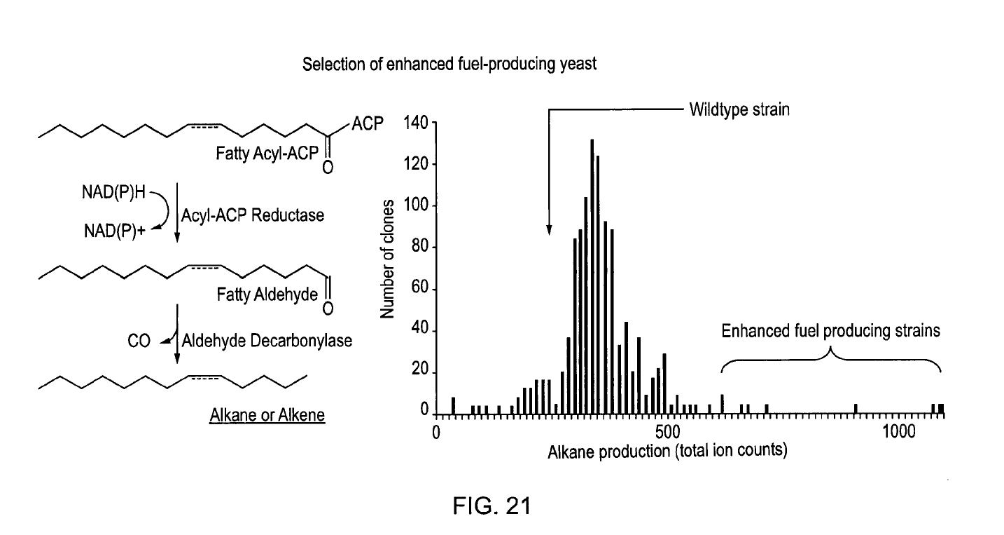

FIG. 21. Selection of enhanced fuel-producing yeast

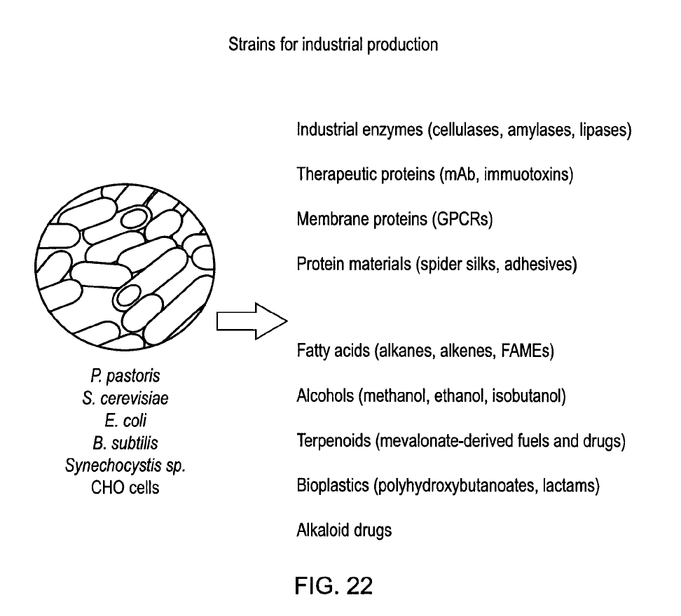

FIG. 22. Strains for industrial production

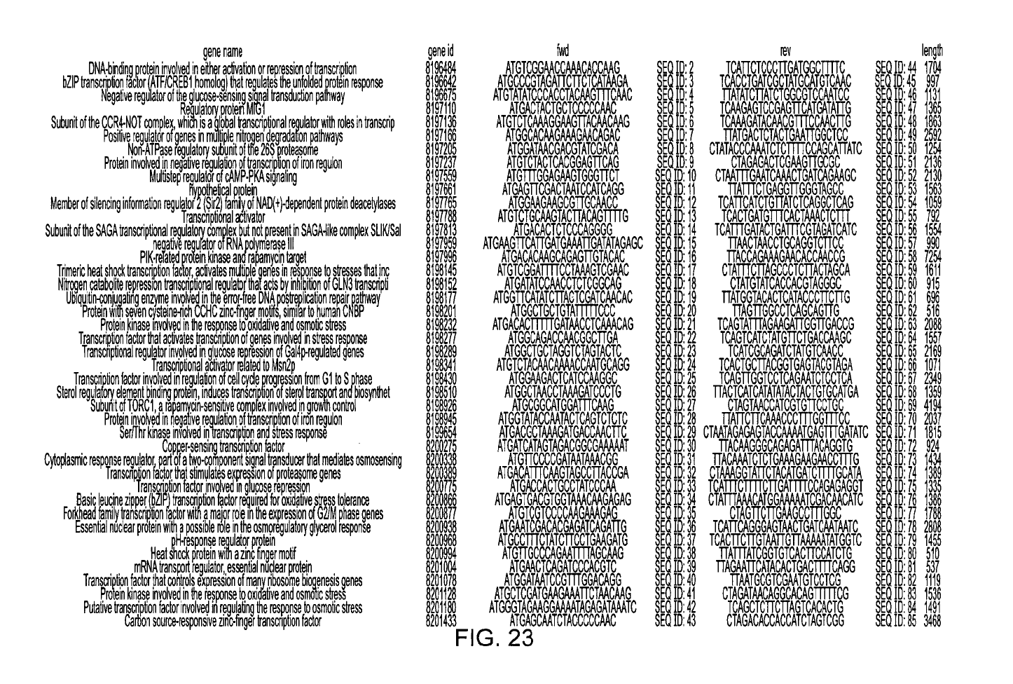

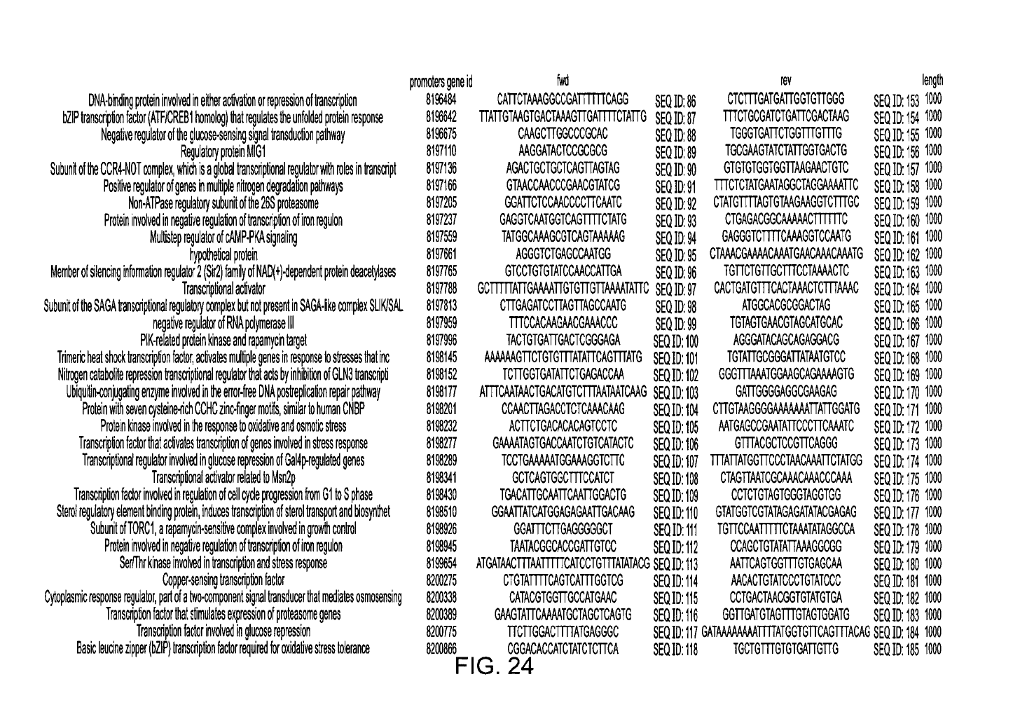

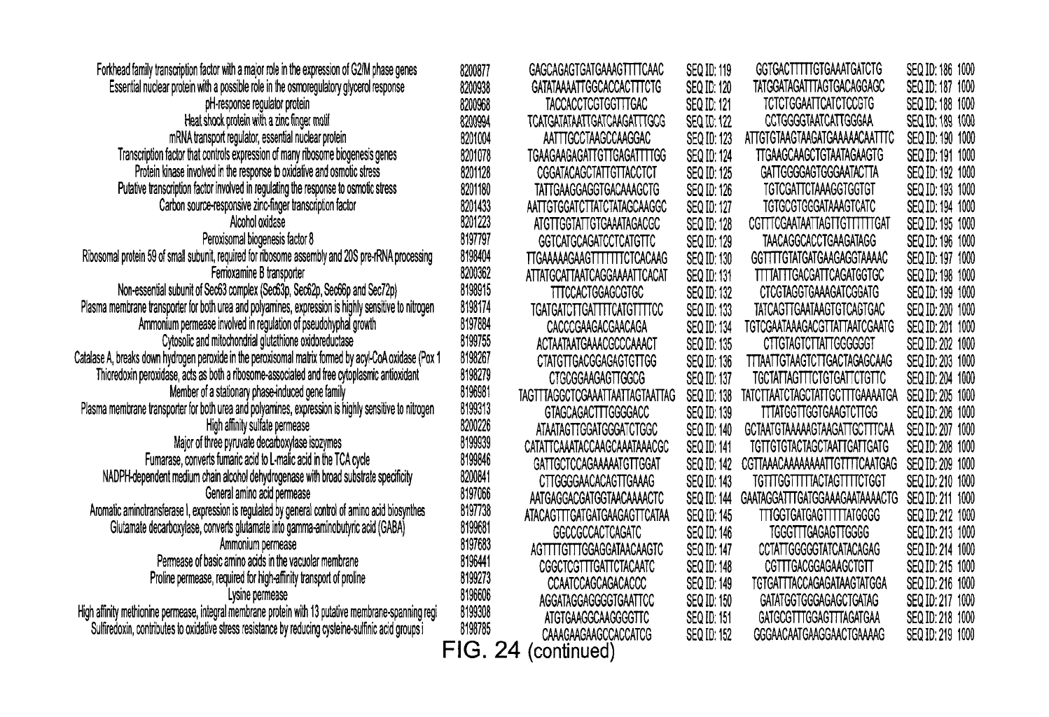

FIG. 23. Example 1 Table 1: P. pastoris regulatory genes and promoters

### EXAMPLE 1

### Rewiring Genetic Networks for Tailored Protein Production Strains

During the 1970s the Phillips Petroleum corporation in conjunction with the Salk Biotechnology/Industrial Associates Inc. developed the methylotrophic yeast Pichia pastoris as a heterologous protein expression platform. This expression system has several attributes that promotes its use for large scale heterologous protein production. To begin with the P. pastoris is capable of achieving very high cell densities, up to 130 g/l dry cell weight, in fermentation culture vessels. Additionally protein expression can be tightly controlled through a diauxic from glucose or glycerol onto methanol. This is achieved by placing the heterologous sequence under the control of the promoter of the Alcohol oxidase 1 (AOX1) gene involved in oxidation of methanol during its utilisation as a carbon source. This promoter is strongly and dominantly repressed in the presence of glucose and glycerol but very strongly activated in the presence of methanol and absence of the two prior carbon sources (Egli 1980, Tschopp 1987). Also its native transcript can account for 5% of observed mRNA pool in the presence of methanol as the sole carbon source (Cregg 2000). This strict powerful inducible system allows for sufficient biomass generation prior to gene expression which is likely to have a deleterious effect on growth. As a eukaryote P. pastoris is also capable of complex post translational modification that prokaryote expression factories are unable to provide such as folding, lipidation, disulphide bond formation and glycosylation (Cregg 2000). Strains have now been developed that perform humanised N-glycosylation as opposed to high mannose glycosylation typical of yeasts (Hamilton 2007). Finally the relatively low amount of secreted native protein makes secreted product substantially easier to purify (Cregg 2000).

Despite these promising characteristics system level understanding of this yeast's genomic, proteomic and metabolic control and function is very limited compared to other yeast species such as Saccharomyces cerevisiae. Indeed full genome sequences have only recently been made available publicly (Mattanovich 2009, DeSchutter 2009, Küberl 2011)

### Materials and Methods

### Heterologous Protein Synthesis

Sequences of heterologous proteins for expression in P. pastoris were synthesised by DNA 2.0 using codon preferences for P. pastoris.

Library Construction in E. coli

Library components were PCR amplified from P. pastoris GS115 genomic DNA using primers OW001 to OW220 detailed in Table Primers. Primers were designed to amplify 1000 bp of selected promoters, while a second set were designed to amplify open reading frames (ORFs). Selected promoters and ORFs are detailed in Table Library. Library components were amplified separately. A second PCR was performed to provide an overlapping 5′ region on promoter sequences for Gibson assembly using primer OW272 (Table Primers). An assembly PCR was performed to fuse ORF constructs to a downstream AOX1 3′ terminator fragment amplified using primers OW221 and OW274.

The library was built using an 8 hr isothermal Gibson assembly (Gibson et al., 2009) integrating library components into the Sma1 site of the modified pAO815 expression vector (Invitrogen) (ExpressionVector.fna) as depicted in Figure Expression vector. Proteins chosen for heterologous expression were cloned into the EcoRI site of this vector. Sequences for the different protein sequences cloned into this site are detailed in ExpressedProteins.fna. Note Kozak sequences AAAATGTCT (SEQ ID Number: 1) and Saccharomyces cerevisiae M-α secretion tags where applicable.

### Yeast Stains Transformation, Growth and Maintenance

For media specification please refer to the Invitrogen Multi-Copy Pichia Expression Kit manual.

The his4-strain GS115 used for transformations was maintained on YPD. Electrocompetent cells used for transformations were prepared according to Lin-Cereghino et al. (2005) with cells being resuspended in a 0.01 final volume of BEDS solution. Competent cells were transformed with ˜100 ng of SalI or StuI linearised vector in 0.1 cm electroporation cuvettes using the standard fungal settings on a Bio-Rad MicroPulser Electroporator. Cells were recovered in 1M Sorbitol for 1 hr then plated onto RDB agar plates.

Colonies from plates were picked and placed into individual wells in 96 well plates containing 200 μl of MGY and grown at 30° C. at 700 rpm in a Mictrotron (Infors) for 48 hrs. Glycerol stocks were then prepared by adding 75 μl of 60% (v/v) Glycerol followed by a 30 min incubation at 30° C. prior to storage at −80° C.

To prepare samples for methanol induction glycerol stocks were replica plated into 200 μl of MD and grown for ˜36 hrs. Cells were then spun down for 5 min at 1500 g, the supernatant removed and washed once with 150 μl of MM centrifuged a second time and then resuspended in 200 μl volume of MM. Cultures were subsequently grown for 24 hrs prior to taking GFP fluorescence and OD₆₀₀ to estimate protein expression and fungal growth.

### Quantifying Protein Production and Identifying Outliers

GFP fluorescence (excitation 485 nm emission 510 nm) and OD₆₀₀ were read in optically clear bottomed 96 well plates (Greiner bio-one Cat#655096) on a POLARstar Omega (BMG Labtech). For Libraries GFP fluorescence was normalised by dividing by OD₆₀₀ for each well. In order to make readings comparable across plates OD₆₀₀ normalised fluorescence signals were mean centred and standardised by subtracting the mean and dividing by the standard deviation on an individual plate basis. Samples which showed expression greater than two standard deviations from the mean were assayed a second time.

### Confirming Library Expression Outliers

Clones which showed robust up-regulation following a second screen were PCR cloned into their parent library vector and sequenced. 40 clones were compared to 40 clones of a no library control (i.e. only heterologous expression protein) using a Student's paired two-tailed t-test assuming equal variance using a 0.05 p-value selection threshold (FIG. 1 Primary Screen, Table Sequencing).

### Results

### Primary Library Screens

The aim of our approach was to identify rewired transcriptome networks within the yeast P. pastoris with an altered capability to induce and express genes driven by the AOX1 promoter following a diauxic shift from a glucose carbon base to methanol. 2881 novel transcriptome networks were created by randomly combining 43 open reading frames of transcriptional regulators and placing these under the control 67 promoters of genes involved in signalling, transport, carbon metabolism as well as the promoters of the 43 ORFs (FIG. 2 Library and FIG. 23—Example 1 Table 1).

Genes for four proteins: human insulin, a synthetic immunotoxin composed of diphtheria toxin from Corynebacterium diphtheriae fused to anti-human CD4, human mu-opiod receptor, and a human short fragment chain antibody (ScFv8) that recognises crototoxin from the South American rattle snake Crotalus durissus terrificus were synthesised by DNA 2.0 with codon optimisation for expression in P. pastoris (Elch et al 2011?). A fifth gene encoding spider dragline silk from Nephila clavipes was obtained from the natural source. The genes were cloned into a the EcoRI site of a modified pAO815 vector (Invitrogen) downstream in frame with a S. cerevisiae Ma secretion tag (with the exception of the mu-opiod receptor which possessed an internal membrane localisation signal) and upstream in frame with a yeast codon optimised GFP sequence (Figure Library). GFP with no secretion signal was also cloned into this site. These genes were chosen to in order to challenge the library to discover potential solutions that currently limit the efficiency of protein expression. Proteins were chosen for their large size (silk and Immunotoxin), highly repetitive structure (silk), complex post translational modifications (Immunotoxin and ScFv8), cell toxicity (Immunotoxin) and cell membrane localisation (opioid receptor). GFP and Insulin were chosen as positive controls as they have been successfully been expressed to relatively high levels in P. pastoris.

Library components were cloned into the SmaI site of the same plasmid. These six libraries were screened for clones which exhibited enhanced GFP expression at levels greater than two standard deviations above the mean of the library. Strains with potential enhanced expression were tested a second time.

### Secondary Screen Validation

Strains showing robust increased expression relative to no library controls were sequenced. Unique rewired sequence constructs (Table Sequencing) were PCR and cloned. GFP expression was compared to no library controls with a t-test using a 0.05 p-value selection threshold in a 40 clone comparison (FIG. 1 Primary Screen, Table Sequencing). Importantly, initial screens identified a number of library isolates which show significantly enhanced protein expression. Additionally, certain constructs were isolated numerous times in different libraries and subsequently validated. This not only strengthens the argument that such constructs are having a physiological beneficial effect on protein expression but also reveals that certain constructs may be broadly advantageous to heterologous protein expression in general. Conversely we would also expect certain constructs to favour expression of proteins with particular properties such as size, repetitive sequence and complex post-translational modification.

Table Sequencing: Promoter and ORF combinations identified and
verified using a t-test comparing 40 clones with respect to different
expressed proteins.

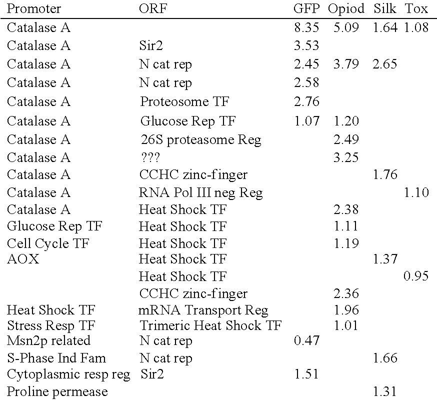

Purple indicates absence of promoter or ORF in the sequences strain. Grey components
indicate isolated homologous to library targets cloned due to non-specific annealing of
primers. Numbers in indicate fold enhancement in expression relative to no rewiring
control. p-value: red > 0.05, 0.05 < yellow < 0.001, green < 0.001.

A range of different rewiring constructs were identified, while promoter and ORF components showed enrichment for functions like stress response, mRNA regulation (in addition to transcription factors) and nutrient regulation. Components such as the CatA promoter (8198267), Heat Shock Factor TF (8200994) and Nitrogen catabolite Repressor ORFs (8198117), were repeatedly isolated in conjunction with different components suggesting a dominant effect on enhanced protein expression. In particular the Cat A promoter and the Heat Shock Factor ORF along with the proline permiase promoter (8199273) and a CCHC zinc finger ORF (8198201) were isolated in absence of an appropriate rewiring partner. This may indicate that simple over expression of these identified ORFs is sufficient to improve heterologous protein expression, however fine tuning temporal ORF expression through rewiring may help to further improve heterologous expression. The isolation of orphaned promoter regions is less clear, perhaps the duplication of these regions in the genome results in titration of limiting regulatory factors culminating in the observed phenotype. Alternatively the 1000 bp promoter regions selected may contain ORFs from genes located upstream. This is indeed a possibility for the Cat A promoter which contains a truncated ORF from an 18S rRNA maturation factor (8198708) with an in frame start-codon.

### Broad Acting Isolate

The orphaned Cat A promoter shown in FIG. 3 18S Mat was investigated with respect to expression of all six different heterologous proteins. Importantly all six proteins showed significantly enhanced expression when expressed alongside this construct. GFP, Insulin and ScFv8 three, shorter, simpler sequences showed around a six fold enhancement in expression, while the remaining larger and proteins showed lower improvements but still statistically significant.

### Combinatorial Rewiring and Improvements

The spider-silk sequence is highly repetitive containing a high number of glycine (43.3%) and alanine (33.8%) residues, with this repetition being primarily encoded by two dominant codons GGA and GCA respectively. Spiders exhibit adjusted tRNA pools in their silk glands with higher levels of Gly-tRNAs and Ala-tRNAs (Candelas et al 1990). In an attempt to replicate this environment the Pichia tRNA pool was adjusted by placing selected tRNA genes downstream of the AOX1 promoter. tRNAs were chosen by screening the P. pastoris genome for putative tRNA genes using Lowe and colleagues tRNA prediction algorithm (Chan and Lowe 2009). Three tRNAs with the appropriate Gly-UCC anti-codon and two for the Ala-UGC anti-codon were identified. These five genes were assembled into a Gly-Ala-Gly-Ala-Gly sequence using IDT g-blocks and Gibson assembly and placed downstream of an AOX1 promoter. This construct showed improved silk expression and this had an additive effect when coupled with the Orphaned Cat A promoter with the combinatorial re-wiring showing higher level expression compared to either construct alone. FIG. 4 Silk Production.

### Example 2

### Enhancing Protein and Metabolite Production in Microbial Systems by Rewiring Genetic Control

### SUMMARY

The use of microbes to produce therapeutics, materials, chemicals, and fuels is an area of intense academic and industrial interest, and has the potential to transform market sectors as diverse as healthcare, agriculture, and energy. Modern metabolic engineering generally involves expression of heterologous proteins and metabolic pathways in a fast growing, genetically tractable host such as the bacteria E. coli or yeast S. cerevisiae. However, productivity and yields of desired protein or metabolite products are often low. The optimisation of microbial strains to overproduce a product of interest is a major focus of metabolic engineering and synthetic biology, which primarily consists of flux balance modelling paired with gene knockouts or overexpression.

Here, we have developed a novel approach engineering microbial strains that show enhanced metabolite or protein production. The genetic regulatory network of microbial organisms has evolved to maximise growth and survival in a variety of dynamic environmental conditions found in Nature. However, many of these responses can be suboptimal in terms of industrial manufacture of valuable bioproducts. We have constructed a library of transcription factor open reading frames (ORFs) under the control of regulatory promoters from different genes. These synthetic promoter-ORF constructions act as rewiring elements when introduced into the cell: for example, environmental conditions that induce the expression of a given gene (in the wildtype organism) now also induce the expression of an unrelated regulatory gene. This has the effect of randomly placing links between different nodes of the network, thereby rewiring the way in which the cell processes information about its environment and cellular state (FIG. 5).

### Results: Protein Overexpression

Here, we have found that rewiring the transcriptional control network of the cell yields strains with enhanced protein or metabolite production capacity. We first identified regulatory gene open reading frames and use combinatorial DNA assembly strategies to construct vectors for rewiring the P. pastoris gene network. We used gene ontology classification to search the P. pastoris genome sequence for retulatory genes. We then constructed a library using these genes as well as transcription factors implicated in control of ribosome biogenesis, stress response, stationary phase, heat shock, and the unfolded protein response (FIG. 6, Table 1).

The genes and promoters identified above were amplified from the P. pastoris genome. We used an isothermal overlap reaction for rapidly assembling multiple large fragments of DNA to combine promoters, ORFs and a Pichia vector backbone. (FIG. 6). The vector backbone is designed to integrate into the genome. This isothermal method has been used to assemble genetic pathways and entire bacterial genomes. ORFs and promoters can be combinatorially assembled by including flanking common overlap sequences. The resulting library is transformed into P. pastoris.

The library of rewired promoter-ORF pairs was transformed into a P. pastoris strain expressing GFP under control of the highly methanol inducible AOX promoter. Cells were grown and induced in deep well 96-well plates. The library was induced with methanol and screened for fluorescence. We assayed approximately 5000 individual colonies, providing 2× coverage of the initial library diversity. A sample of the data is shown in FIG. 7. This data is normalised by subtraction of the population mean and division by the standard deviation to compare distributions across plates.

The selected overexpression strains can be subjected to further rounds of rewiring and selection. It is considered that multiple rewirings of the regulatory network will result in increased protein production. This is accomplished by inserting the promoter-ORF library in the existing selected vector using a modified version of the assembly protocol. This new library is screened and characterised as above. Additional rounds of diversification and screening generated populations that show significantly enhanced productivity.

### Results: Alkane Production

We used the same initial library to identify strains that overproduce long-chain alkanes in P. pastoris. We placed a fatty-acyl ACP reductase under the control of the AOX promoter. This gene, when expressed with a fatty aldehyde decarbonylase, is able to use fatty acid biosynthetic precursors to produce fuel range alkanes (FIG. 8). Production can be screened via luminescence, as the aldehyde intermediate is a good substrate for bacterial luciferase (luxAB).

FIG. 9 shows the results from an additional “round” of library generation and screening. The rewiring library was constructed in two clones showing high alkane productivity from a first round of screening. Thus, these populations have two rewiring constructs in the genome.

### Further Efforts

We have applied the rewiring optimisation scheme to the following protein products, for example as described in Example 1: human insulin, human opioid receptor, spider silks, a diphtheria-antibody immunotoxin, and a monoclonal antibody fragment.

In addition, the scheme can be used to engineer strains for the overproduction of lysine, glutamine, alcohols, s-adenosylmethionine, and mevalonate, which are all precursors for commodity or fine chemicals.

Artificial rewired networks which exhibit enhanced protein productivity can be assessed using high-resolution time series expression profiling of transcriptional regulators using real time PCR spanning the period of protein production. Dynamic Bayesian network inference can then be used to model the rewired network to observe the effect that novel edge incorporation has had on network structure. This learned data can be used to inform future attempts to improve productivity of expression hosts.

### Methods

Media and Transformation of P. pastoris Strain GS115

Minimal Glycerol Medium (MG), Minimal Methanol Medium (MM) and Regeneration Dextrose Biotin (RDB) agar plates were prepared according to the Pichia Expression Kit manual (Invitrogen).

P. pastoris strain GS115 cells were made electrocompetent by treatment of cultures with BEDS (10 mM bicine-NaOH pH 8.3, 3% ethylene glycol, 5% dimethyl sufoxide and 1M sorbitol) and dithiothreitol (100 mM). Electrocompetent cells were transformed using 60 ng of linearised plasmid digested with StuI (NEB) using a Gene Pulser® electroporator. Cuvettes with 1.0 mm gap were used and the settings for electroporation as follows: 1500 V charging voltage 200Ω resistance and 25 μF capacitance. Cells were plated in RDB plates for selection of His+ transformants and grown at 30° C. until visible growth of colonies.

### Cloning

A chemically synthesised and E. coli optimised version of the short-chain fatty acid reductase PCC7942_orf1593 reported by Schirmer et al (2010) as producing fatty-aldehyde intermediates in a pathway for biosynthesis of alkanes was synthesised by DNA 2.0 and is considered the gene of interest (GOI).

A combinatorial library of promoters and transcription factors using pAO815 (Invitrogen) as vector backbone was used as base for the expression of the GOI. Cloning proceeded with the insertion of the GOI in the EcoRI site of the vector, making the expression of the gene methanol inducible.

### Growth and Induction

His+ transformants were transferred to 96-well microplates with 200 μl of MG medium and grown for 48 hours, washed with 200 μl of MG medium and re-suspended with 200 μl of MM medium for induction for 24 h.

### Luciferase Assay

E. coli membranes are permeable to aldehyde diffusion, and so plasmid expressing the bacterial luciferase genes LuxA and LuxB serve as efficient reporters for the presence of these molecules in aqueous solution.

Centrifugation followed culture induction, with 150 μl of supernatant being transferred to 96 well Nunc fluorescence plates. Addition of 50 μl of culture of the E. coli reporter strain preceded fluorescence readings using a POLARstar Omega plate reader in continuous mode for 16 minutes.

Cultures were resuspended with 150 μl of MM medium and 100 μl was transferred to 96-well Greiner Fluotrac plates for OD600 measurements using a BioTek Synergy HT-I.

### Data Analysis

Fluorescence and luminescence signal for each sample was normalised by OD600 and maximum luminescence values were used as reference value for expression. Values for each individual plate were statistically normalised for outlier identification.

1. A method of identifying a cell with increased production of a desired product, the method comprising:
(a) providing a population of cells which produce the desired product;
(b) introducing a plurality of distinct nucleic acid molecule species into the population of cells, wherein the nucleic acid molecules comprise a first region which is operably-linked to a second region, wherein
the first region comprises a promoter region from a first gene, and
the second region comprises a nucleic acid sequence from a second gene that is capable of expressing a regulatory product under control of the promoter region;
(c) culturing the population of cells under conditions in which the cells express the regulatory product under control of the promoter region; and
(d) testing the population of cells for production of the desired product, thereby to identify a cell or cells with increased production of the desired product.

2. A method according to claim 1, the method further comprising isolating a cell or cells with increased production of the desired product.

3. A method according to claim 2, wherein the method further comprises repeating steps (b), (c) and (d) on the isolated cells or cells, or descendants thereof with increased production of the desired product, thereby to identify a cell or cells with further increased production of the desired product.

4. A method according to claim 3, the method further comprising isolating a cell or cells with further increased production of the desired product.

5. A method according to any of claims 1 to 4 wherein the desired product is not encoded by the introduced nucleic acid molecule.

6. A method according to any of claims 1 to 5, wherein the plurality of distinct nucleic acid molecule species comprises a plurality of combinations of multiple first and multiple second regions.

7. A method according to any of claims 1 to 6 wherein the expressed regulatory product is a polypeptide.

8. A method according to any of claims 1 to 6 wherein the expressed regulatory product is an RNA molecule.

9. A method according to any of claims 1 to 8, wherein the method further comprises isolating the nucleic acid molecule(s) introduced into the cell or cells with increased production of the desired product.

10. A method of producing a cell with increased production of a desired product, the method comprising:
providing a cell which produces a desired product; and
introducing into the cell a nucleic acid molecule that has been isolated according to claim 9.

11. A method according to any of claims 1 to 8, wherein the method further comprises identifying the first and second regions of the nucleic acid molecule(s) introduced into the cell or cells with increased production of the desired product.

12. A method of producing a cell with increased production of a desired product, the method comprising:
providing a cell which produces a desired product; and
introducing into the cell a nucleic acid molecule comprising a promoter region operably-linked to a nucleic acid sequence that is capable of expressing a regulatory product under control of the promoter region, wherein the promoter region, and the nucleic acid sequence that is capable of expressing the regulatory product, have been identified according to the method of claim 11.

13. A method of producing a desired product, the method comprising:
culturing a cell with increased production of the desired product that has been produced, identified or isolated according to the method of any of claims 1 to 12, or the descendants thereof with increased production of the desired product, under conditions in which the cell expresses the desired product.

14. A method according to claim 13, the method further comprising recovering the desired product from the cells or the cell culture.

15. A method according to claim 13 or 14, the method further comprising isolating, concentrating, purifying and/or formulating the desired product.

16. The method according to any of claims 1 to 15 wherein the cell is a prokaryotic cell.

17. A method according to claim 16 wherein the prokaryotic cell is a cell listed in Table 1.

18. A method according to claim 17 wherein the prokaryotic cell is a Bacillus or Escherichia or Synechocystis.

19. A method according to claim 17 or 18 wherein the prokaryotic cell is B. subtilis or E. coli.

20. A method according to any of claims 17 to 19 wherein the regulatory polypeptide is a transcription factor or a sigma factor

21. A method according to any of claims 17 to 19 wherein the regulatory RNA molecule is a microRNA

22. A method according to any of claims 1 to 15 wherein the cell is a eukaryotic cell.

23. A method according to claim 22 wherein the eukaryotic cell is a fungal cell, an insect cell, a plant cell, an algal cell or a mammalian cell.

24. A method according to 23 wherein the fungal cell is a yeast.

25. A method according to 23 or 24 wherein the fungal cell is P. pastoris or S. cerevisiae or a fungal cell as set out in Table 2.

26. A method according to 23 wherein the plant cell is an Arabidopsis thaliana, Nicotiana tabacum, Zea mays, soybean Glycine max, Brassica, Helianthus, Gossypium, Medicago, Triticum, Hordeum, Avena, Sorghum or Orya cell.

27. A method according to 23 wherein the mammalian cell is a human, mouse, rat, rabbit, bovine or dog cell.

28. A method according to 27 wherein the mammalian cell is a CHO cell.

29. A method according to any of claims 22 to 28 wherein the regulatory polypeptide is a transcription factor.

30. A method according to claim 29 wherein the regulatory polypeptide is listed in Table 3 or is a homolog of a regulatory polypeptide listed in Table 3.

31. A method according to any of claims 22 to 28 wherein the regulatory RNA molecule is a microRNA.

32. A method according to any of claims 1 to 31 wherein the desired product is an industrial enzyme, a therapeutic protein, a membrane protein, a functional protein material, a fatty acid, an alcohol, a terpenoid, a bioplastic, a pigment, or an alkaloid drug.

33. A method according to any of claims 1 to 32 wherein the desired product is a growth factor, cellulase, an amylase, a lipase, an antibody or an antibody fragment, an immunotoxin, a GPCR, a spider silk, an adhesive protein, an alkane, an alkene, a FAME, methanol, ethanol, isobutanol, a mevalonate-derived fuel, a mevalonate-derived drug, a polyhydroxybutyrate, a polyhydroxybutanoate, a lactam, or lycopene.

34. A method according to any of claims 1 to 33 wherein the cell is from Pichia ssp or P. pastoris and the desired product is a human opioid receptor, insulin, an antibody fragment, a spider silk polypeptide or an immunotoxin therapeutic.

35. A method according to any of claims 1 to 32 wherein the desired product is not Green Fluorescent Protein (GFP).

36. A method according to any of claims 1 to 15 wherein the cell is P. pastoris, the desired product is silk protein, the first region comprises a promoter from a P. pastoris gene encoding lysine permease protein 8196606, and the second region comprises a nucleic acid sequence that encodes P. pastoris protein 8198201; or wherein the first region comprises a promoter from a P. pastoris gene encoding Catalase A (8198267), and the second region comprises a nucleic acid sequence that encodes P. pastoris Nitrogen catabolite Repressor ORF (8198117).

37. A method according to any of claims 1 to 15 wherein the cell is P. pastoris, the desired product is an scFv antibody, the first region comprises a promoter from a P. pastoris gene encoding protein 8197996, and the second region comprises a nucleic acid sequence that encodes P. pastoris protein 8200775; or wherein the first region comprises a promoter from a P. pastoris gene encoding encoding Catalase A (8198267).

38. A method according to any of claims 1 to 15 wherein the cell is P. pastoris, the desired product is insulin, the first region comprises a promoter from a P. pastoris gene encoding protein 8198945, and the second region comprises a nucleic acid sequence that encodes P. pastoris protein 8198341; or wherein the first region comprises a promoter from a P. pastoris gene encoding encoding Catalase A (8198267).

39. A cell produced by, identified by, or isolated by the method of any of claims 1 to 12 or 16 to 38, or a descendant thereof, with increased production of the desired product.

40. A method for increasing the production of a desired product, the method comprising:
producing, identifying, or isolating, by the method of any of claims 1 to 12 or 16 to 39, cells with increased production of the desired product;
culturing the cells, or descendants thereof with increased production of the desired product, under conditions in which the cells express the desired product; and
recovering the increased amount of the desired product from the cells or the cell culture.

41. A method according to claim 40, the method further comprising isolating, concentrating, purifying and/or formulating the increased amount of the desired product.

42. A method for bioremediation of a waste product, the method comprising:
providing cells produced by, identified by, or isolated by the method of any of claims 1 to 12 or 16 to 35, or descendants thereof, with increased production of a desired product that metabolises the waste product; and
exposing the cells to the selected waste product,
thereby providing bioremediation of the selected waste product.

43. A collection of cells that produce a desired product, wherein the cells in the collection comprise a plurality of distinct nucleic acid molecule species,
wherein each nucleic acid molecule comprises a first region which is operably-linked to a second region, wherein the first region comprises a promoter region from a first gene, and the second region comprises a nucleic acid sequence from a second gene that is capable of expressing a regulatory product under control of the promoter region, and
wherein the desired product is not GFP.

44. A collection of cells according to claim 43 wherein the plurality of distinct nucleic acid molecule species comprises a plurality of combinations of multiple first and multiple second regions.

45. A collection of cells according to claim 43 or 44 wherein the nucleic acid molecules are integrated into the genome of the cells that produce the desired product.

46. A collection of cells according to claim 43 or 44 wherein the nucleic acid molecules are maintained extra-chromosomally in the cells that produce the desired product.

47. A collection of cells according to any of claims 43 to 46 wherein the cells are Pichia pastoris cells.

48. A kit of parts comprising:
a population of cells that produce a desired product; and
a plurality of distinct nucleic acid molecule species, wherein each nucleic acid molecule comprises a first region which is operably-linked to a second region, wherein
the first region comprises a promoter region from a first gene, and
the second region comprises a nucleic acid sequence from a second gene that is capable of expressing a regulatory product under control of the promoter region in the cells that produce the desired product.

49. A kit of parts according to claim 48 wherein the plurality of distinct nucleic acid molecule species comprises a plurality of combinations of multiple first and multiple second regions.

50. A kit of parts according to claim 48 or 49 wherein the desired product is not encoded by the nucleic acid molecule species.

51. A kit of parts according to any of claims 48 to 49 wherein the plurality of nucleic acid molecule species are contained in expression vectors.

52. A kit of parts according to any of claims 48 to 51 wherein the plurality of nucleic acid molecule species are contained in integration vectors that are capable of integration into the genome of the cells that produce the desired product.

53. A kit of parts according to any of claims 48 to 51 wherein the plurality of nucleic acid molecule species are contained in episomal vectors that are capable of extra-chromosomal maintenance in the cells that produce the desired product.

54. A kit of parts according to any of claims 48 to 53 wherein the cells are Pichia pastoris cells.
